(19) United States
(12) Patent Application Publication
(10) Pub. No.: US 2024/0044895 A1
(43) Pub. Date: Feb. 8, 2024

(54) IDENTIFICATION OF SARS-COV-2 EPITOPES DISCRIMINATING COVID-19 INFECTION FROM CONTROL AND METHODS OF USE

(71) Applicant: WISCONSIN ALUMNI RESEARCH FOUNDATION, Madison, WI (US)

(72) Inventors: Irene ONG, Fitchburg, WI (US); David O’CONNOR, Madison, WI (US); Miriam SHELEF, Madison, WI (US); Anna HEFFRON, Madison, WI (US); David BAKER, Northbrook, IL (US); Maya AMJADI, Madison, WI (US); Sean MCILWAIN, Madison, WI (US); Saniya KHULLAR, West Windsor, NJ (US)

(21) Appl. No.: 18/245,868

(22) PCT Filed: Sep. 20, 2021
(86) PCT No.: PCT/US2021/051143

§ 371 Date: Mar. 17, 2023

Related U.S. Application Data
(60) Provisional application No. 63/080,568, filed on Sep. 18, 2020.
(60) Provisional application No. 63/083,671, filed on Sep. 25, 2020.

(51) Int. Cl. G01N 33/569; G01N 21/76; G01N 33/543; C07K 14/165; A61K 39/215

(52) U.S. Cl. CPC G01N 33/56983; G01N 21/76; G01N 33/543; C07K 14/165; A61K 39/215; G01N 2333/165

(57) ABSTRACT

The present invention is directed to peptides for use in the detection of antibodies against SARS-CoV-2, which are indicative of past SARS-CoV-2 infections. Additionally, assays and methods of distinguishing patients having had a prior infection from those vaccinated patients are also provided. Additionally, vaccine compositions for use in eliciting anti-SARS-CoV-2 immune response are provided along with methods of producing antibodies and methods of eliciting an immune response.

### CROSS-REFERENCE TO RELATED APPLICATIONS

This application claims priority to U.S. Application No. 63/080,568 filed on Sep. 18, 2020 and U.S. Application No. 63/083,671, filed on Sep. 25, 2020, the contents of each are incorporated by reference in their entireties.

### STATEMENT REGARDING FEDERALLY SPONSORED RESEARCH

This invention was made with government support under OD017850 and HHSN272201600007C awarded by the National Institutes of Health. The government has certain rights in the invention.

### SEQUENCE LISTING

Sequences can be found in Table 1 and 2 and in the Tables in the Figures and are incorporated herein by reference in its entirety.

### BACKGROUND

The immune response to severe acute respiratory syndrome coronavirus 2 (SARS-CoV-2) infection remains poorly understood. Protective antibodies have been posited for some coronaviruses, such as Middle Eastern respiratory syndrome coronavirus (MERS-CoV) and severe acute respiratory syndrome coronavirus (SARS-CoV), while antibody-dependent enhancement has been documented for other coronaviruses, such as feline infectious peritonitis. SARS-CoV-2, the cause of the pandemic illness of coronavirus infectious disease 2019 (COVID-19), is the most recent coronavirus to emerge as a human pathogen, and has caused at least 4.68 million deaths worldwide since December 2019. Yet, our understanding of the breadth and implications of the anti-SARS-CoV-2 antibody response remains limited.

Antibody responses against two proteins in SARS-CoV-2, the spike (S) or surface protein and the nucleocapsid (N) protein, have been documented. All serology-based testing platforms in production to date use one or both of these proteins as antigens and have sensitivities ranging from 79.6% to 91.7% at least a month after symptom onset. Little is known about antibody responses to the virus' other structural and non-structural proteins, including the membrane (M) protein, which is the most abundant protein in the virion, or the accessory proteins in the virus' largest open reading frame (i.e., orflab).

SARS-CoV-2 infection results in highly variable outcomes, ranging from asymptomatic infections to hospitalizations, intubations, and death. Differences in the immune response are thought to contribute to these differences in outcomes, but this, too, is not well-defined. Further, little is known about the cross-reactivity of anti-SARS-CoV-2 antibodies with other coronaviruses.

Thus, there remains a desperate need in the art for a better understanding of the humoral response against SARS-CoV-2. This understanding will play a critical role in the development of appropriate diagnostics, vaccines, and medical countermeasures.

### SUMMARY OF INVENTION

In the present disclosure, the inventors used an ultra-dense peptide microarray to profile antibody binding throughout the full SARS-CoV-2 proteome in severely and mildly ill patients. Additionally, they used this microarray to profile cross-reactive binding of anti-SARS-CoV-2 antibodies to other coronaviruses. In one aspect, the disclosure provides a method of detecting the presence of SARS-CoV-2 antibodies in a sample, the method comprising: contacting the sample with one or more peptide selected from SEQ ID NO:1-16 and Tables 1, 2, 6, 9-12 or a peptide having at least 90% sequence similarity to a peptide of SEQ ID NO:1-16 or in Tables 1, 2, 6, 9-12; and detecting the binding of one or more antibodies from the sample to the one or more peptide by a detection agent, wherein the presence of the one or more antibodies within the sample indicates the presence of antibodies against SARS-CoV-2. In some aspects, the presence of antibodies signifies a past SARS-CoV-2 infection. In some embodiments, the detecting of the antibodies can distinguish between a subject having had SARS-CoV-2 and a vaccinated subject, as described herein. In some aspects, the detecting of the antibodies can distinguish a person either vaccinated or having had CoV-2 from a naïve subject. In other aspects, the detecting of the antibodies can distinguish between a subject having had SARS-CoV-2 and a vaccinated subject, or a subject that has had neither.

In another aspect, the disclosure provides an immunoassay for detecting antibodies to SARS-CoV-2 in a biological sample, the assay comprising: a capture agent comprising one or more peptides selected from SEQ ID NO:1-10 and Tables 1, 2 and 9-12 or a peptide with at least 90% similarity to one or more peptides in Tables 1. 2 and 9-12, and a detection agent capable of binding to the one or more antibodies that bind to the capture agent.

In a further aspect, the disclosure comprises a method of detecting and distinguishing a subject infected with SARS-CoV-2, subject vaccinated and an uninfected individual comprising obtaining a sample from a subject; contacting the sample with the immunoassay described herein, wherein detection of the peptide to the membrane protein and to a spike peptide can distinguish between the subjects that have had SARS-CoV-2 and a subject vaccinated vs a naïve subject.

In another aspect, the disclosure provides a vaccine composition comprising an adjuvant and one or more peptides selected from Tables 1, 2, 6 and 9-12 or a peptide having at least 90% sequence similarity to one or more peptides in Tables 1, 2, 6, and 9-12. In some embodiments, the peptides comprise a 16-mer or longer of a peptide in Tables 1, 2 6, 9-12.

In another aspect, the present disclosure provides use of the vaccine composition described herein for eliciting an immune response in a subject against SARS-CoV-2.

In a further aspect, the disclosure provides a polynucleotide construct comprising a heterologous promoter region and a nucleotide sequence encoding at least one peptide selected from SEQ ID NO:1-16 and Tables 1, 2, 6, and 9-12 or a peptide having at least 90% sequence similarity to one or more peptides of SEQ ID NO:1-16 and in Tables 1, 2, 6, and 9-12.

In another aspect, the disclosure provides a method of eliciting an immune response to SARS-CoV-2 in a subject, the method comprising administering an effective amount of the vaccine composition described herein to the subject to elicit an immune response.

In a further embodiment, the disclosure provides a kit comprising the immunoassay for SARS-CoV-2 as described herein.

In yet another aspect, the disclosure provides a method of detecting the presence of antibodies against a coronavirus in a sample, the method comprising: contacting the sample with one or more peptide selected from Tables 12A-12C, a homologous protein to one or more peptide of Tables 12A-12C, or a peptide having at least 90% sequence similarity to a peptide in Tables 12A-12C; and detecting the binding of one or more antibodies from the sample to the one or more peptide by a detection agent, wherein the presence of the one or more antibodies within the sample indicates the presence of a past the coronavirus infection within the subject.

In yet another aspect, the disclosure provides a method of detecting and distinguishing a subject infected with SARS-CoV-2 or a subject vaccinated comprising obtaining a sample from a subject; contacting the sample with the immunoassay comprising a capture agent comprising at least one peptide of the membrane protein of SEQ ID NO:1 or from Table 2 and a detection agent; wherein detection of the peptide to the membrane protein can distinguish between a subject having had SARS-CoV-2 and a subject vaccinated.

In yet another aspect, the disclosure provides a method of testing a convalescent plasma sample for antibodies to SARS-CoV-2, the method comprising: obtaining a plasma sample; contacting the sample with the immunoassay of any one of claims 11-22, wherein detecting at least one peptide confirms the convalescent plasma contains anti-SARS-Cov2 antibodies.

### BRIEF DESCRIPTION OF THE DRAWINGS

FIG. 1. Patients and controls show reactivity to a poliovirus control. Sera from 20 controls collected before 2019 were assayed for IgG binding to the full proteome of human poliovirus 1 on a peptide microarray. Binding was measured as reactivity that was >3.00 standard deviations above the mean for the log₂-quantile normalized array data. Patients and controls alike showed reactivity to a well-documented linear poliovirus epitope (start position 613 [IEDB.org]; orange shading in line plot). The data used in this analysis can be accessed online at: github.com/Ong-Research/UW_Adult_Covid-19. COVID-19, coronavirus disease 2019: IgG, immunoglobulin G.

FIG. 2. Control sera show reactivity to CCCoVs and to SARS-CoV. MERS-CoV, and SARS-CoV-2. Sera from 20 controls collected before 2019 were assayed for IgG binding to the full proteomes of 9 CoVs on a peptide microarray. Viral proteins are shown aligned to the SARS-CoV-2 proteome with each virus having an individual panel; SARS-CoV-2 aa position is represented on the x-axis. Binding was measured as reactivity that was >3.00 standard deviations above the mean for the log₂-quantile normalized array data. Peptides for which >40% of the controls showed binding are indicated by a black diamond. The data used in this analysis can be accessed online at: github.com/Ong-Research/UW_Adult_Covid-19. aa, amino acid; CCCoVs, “common cold” CoVs; CoV, coronavirus: IgG, immunoglobulin G; MERS-CoV, Middle Eastern respiratory syndrome coronavirus; SARS-CoV, severe acute respiratory syndrome coronavirus; SARS-CoV-2, severe acute respiratory syndrome coronavirus 2.

FIG. 3. Anti-SARS-CoV-2 antibodies bind throughout the viral proteome. Sera from 40 COVID-19 convalescent patients were assayed for IgG binding to the full SARS-CoV-2 proteome on a peptide microarray. B cell epitopes were defined as peptides in which patients' average log₂-normalized intensity (black lines in line plots) is 2-fold greater than controls' (gray lines in line plots), and t test statistics yield adjusted p-values <0.1: epitopes are identified by orange shading in the line plots. Epitopes having at least 100% specificity and at least 80% sensitivity for SARS-CoV-2 infection are indicated by a black arrow. The 1-M-24 epitope, which had the highest combined reactivity, specificity, and sensitivity of all epitopes we defined, is indicated by a black star. The data used in this analysis can be accessed online at: github.com/Ong-Research/UW_Adult_Covid-19. aa, amino acid; COVID-19, coronavirus disease 2019: IgG, immunoglobulin G; SARS-CoV-2, severe acute respiratory syndrome coronavirus 2.

FIG. 4. Anti-SARS-CoV-2 antibodies to S protein show the highest binding in the fusion cleavage site. Binding reactivities were localized on a coordinate file for a trimer of the SARS-CoV-2 S protein using a dark blue (low, 0.00 fluorescence intensity) to red (high, 9.00 fluorescence intensity) color scale. (A) COVID-19 convalescent patients, (B) naïve controls, and (C) the difference between patients and controls are shown. The highest reactivity occurred in the fusion peptide (aa 788-806) and at the base of the extracellular portion of the molecule (aa 984-1163), with lower reactivity in the receptor-binding domain (aa 319-541). (D) Key regions of the S protein are labeled and colored. In the S1 subunit (aa 14-685), the NTD (aa 14-305) is red, and the RBD (aa 319-541) is blue. Within the RBD, the RBM (aa 438-506) is yellow, and the residues that bind to the ACE2 receptor (aa 446, 449, 453, 455-456, 473, 475-476, 484, 486-487, 489-490, 493, 496, 498, 500-502, and 505) are in black. In the S2 subunit (aa 686-1273), the FP (aa 788-806) is green, the HR1 (aa 912-984) is orange, and the base of the extracellular part of the protein (base, roughly an 1140-1160) is purple. The remainder of the protein is gray. The data used in this analysis can be accessed online at: github.com/Ong-Research/UW_Adult_Covid-19. AA, amino acid; ACE2. angiotensin converting enzyme 2; COVID-19, coronavirus disease 2019: FP, fusion peptide; HR1, heptad repeat 1; NTD. N-terminal domain; RBD, receptor-binding domain; RBM, receptor-binding motif; S, spike: SARS-CoV-2, severe acute respiratory syndrome coronavirus 2.

FIG. 5. Anti-SARS-CoV-2 antibodies may cross-react with other CoVs. Sera from 40 COVID-19 convalescent patients were assayed for IgG binding to 9 CoVs on a peptide microarray; averages for all 40 are shown. Viral proteins are aligned to the SARS-CoV-2 proteome; SARS-CoV-2 an position is represented on the x-axis. Regions that may be cross-reactive across all β-CoVs (*) or cross-reactive for SARS-CoV or MERS-CoV (#) are indicated. Gray shading indicates gaps due to alignment or lacking homologous proteins. Cross-reactive binding is defined as peptides in which patients' average log₂-normalized intensity is 2-fold greater than controls' and t test statistics yield adjusted p-values <0.1. The data used in this analysis can be accessed online at: github.com/Ong-Research/UW_Adult_Covid-19. aa, amino acid, β-CoV, betacoronavirus; CoV, coronavirus: COVID-19, coronavirus disease 2019; IgG, immunoglobulin G; MERS-CoV. Middle Eastern respiratory syndrome coronavirus: SARS-CoV, severe acute respiratory syndrome coronavirus; SARS-CoV-2. severe acute respiratory syndrome coronavirus 2.

FIG. 6. Higher IgG binding to SARS-CoV-2 peptides in COVID-19 convalescent patients compared to controls by ELISA. (A) IgG binding to SARS-CoV-2 peptides in COVID-19 convalescent (n=40) and naïve control (n=20) sera was measured by ELISA. Bars indicate mean abs +/− SEM and ****p<0.0001 by t test. (B) Anti-SARS-CoV-2 peptide IgG detected by ELISA was compared to array findings by Spearman rank-order correlation (Spearman correlation coefficient, p) for COVID-19 convalescent (n=40, closed circles) and control (n=20, open circles) sera. The data used in this analysis can be accessed online at: github.com/Ong-Research/UW_Adult_Covid-19. abs. absorbance: COVID-19, coronavirus disease 2019; ELISA, enzyme-linked immunosorbent assay; IgG, immunoglobulin G; SARS-CoV-2, severe acute respiratory syndrome coronavirus 2; SEM, standard error of the mean.

FIG. 7. Disease severity correlates with increased antibody binding in specific SARS-CoV-2 epitopes. IgG reactivity against SARS-CoV-2 epitopes identified by peptide microarray in COVID-19 convalescent patients who were never hospitalized versus intubated patients showed statistically significant increases in reactivity in intubated patients for 11 epitopes. The data used in this analysis can be accessed online at: github.com/Ong-Research/UW_Adult_Covid-19. COVID-19, coronavirus disease 2019; IgG, immunoglobulin G; SARS-CoV-2, severe acute respiratory syndrome coronavirus 2.

FIG. 8. Anti-membrane, but not anti-nucleocapsid or anti-spike receptor binding domain, antibodies distinguish between COVID-19 vaccination and past COVID-19 infection 12 months post-symptom resolution. A. Enzyme-linked immunosorbent assay (ELISA) was used to quantify IgG that binds a biotinylated peptide from SARS-CoV-2 membrane (ITVEELKKLLEQWNLV (SEQ ID NO:1)-K-biotin) and nucleocapsid protein (QTVTLLPAADLDDFSK (SEQ ID NO:3)-K-biotin) with results reported as absorbance (abs). Ig that binds to SARS-CoV-2 spike receptor binding domain (RBD) was detected using Lumit™ Dx SARS-CoV-2 Immunoassay (Promega) with results reported as sample/calibrator (S/C). Sera from the following subjects were tested in all assays: COVID-19 naive subjects collected prior to 2019 (n=60), COVID-19 vaccinated subjects with no known COVID-19 infection (Vax n=21), sera from COVID-19 convalescent patients (SARS-CoV-2 PCR+ at diagnosis) collected ˜5 weeks (5w, n=101), ˜3 months (3m, n=98), ˜6 months (6m, n=94), and ˜12 months (12m) post symptom resolution. By 12 months, 73 convalescent subjects had received at least one COVID-19 vaccine dose (12m Vax) and 16 were unvaccinated (12m Unvax). Black dots represent individual samples and gray bars indicate medians. Dashed lines indicate potential cutoffs for positive antibody detection. Kruskal Wallis test with Dunn's multiple comparisons post-test was used to determine if groups were different as compared to naive (#p<0.0001) or Vax (& p<0.001). Mann Whitney test was used to determine if there was a difference between 12m Unvax and 12m Vax groups (**** p<0.0001 or not significant, ns). B. Receiver operating characteristic (ROC) curves were generated using ELISA absorbance values for anti-membrane or anti-nucleocapsid IgG with sera from naive and 5w COVID-19 convalescent patients. Area under the curve (AUC) and Wald 95% confidence intervals are in parentheses. C. Using the cutoffs in (A), the percent positive (black bars) and percent negative (gray bars) results were calculated for naive sera (n=60), sera from vaccinated individuals with no COVID-19 infection (n=21), and 5w (n=121), 3m (n=115), 6m (n=98), and 12m (n=100) COVID-19 convalescent sera.

FIG. 9. Anti-SARS-CoV-2 antibody binding patterns do not vary with neutralizing titer. Sera from 40 COVID-19 convalescent patients were assayed for IgG binding to the full SARS-CoV-2 proteome on a peptide microarray. B cell epitopes were defined as peptides in which patients' average log₂-normalized intensity (black lines in line plots) is 2-fold greater than controls' (gray lines in line plots) and t test statistics yield adjusted p-values <0.1; epitopes are identified by orange shading in the line plots. Data are grouped by their neutralizing titer. COVID-19, coronavirus disease 2019; IgG, immunoglobulin G; SARS-CoV-2, severe acute respiratory syndrome coronavirus 2.

FIG. 10. Alignment of epitopes in human and animal CoVs for which antibodies in sera from COVID-19 convalescent patients showed apparent cross-reactive binding. Alignments were performed in Geneious Prime 2020.1.2 (Auckland. New Zealand). CoV, coronavirus; COVID-19, coronavirus disease 2019.

FIG. 11 Specificity and sensitivity for past SARS-CoV-2 infection in 40 COVID-19 convalescent patients compared to 20 naïve controls of individual 16-mer peptides comprising epitopes throughout the full SARS-CoV-2 proteome. COVID-19, coronavirus disease 2019; SARS-CoV-2, severe acute respiratory syndrome coronavirus 2. doi.org/10.1371/journal.pbio.3(0)1265.s004

FIG. 12. Epitopes paired with the 1-M-24 epitope obtained AUC-ROC of 1.0 for SARS-CoV-2 infection in 40 COVID-19 convalescent patients and 20 naïve controls using leave-one-out cross validation with linear discriminant analysis. AUC-ROC, area under the receiver operating characteristic curve; COVID-19, coronavirus disease 2019; SARS-CoV-2, severe acute respiratory syndrome coronavirus 2.

FIG. 13. Comparison of antibody binding in SARS-CoV-2. B cell epitopes in 8 intubated COVID-19 convalescent patients compared to 25 symptomatic but never hospitalized COVID-19 convalescent patients compared by multilinear regression accounting for age, sex, immunocompromising conditions, and Charlson comorbidity index score. COVID-19, coronavirus disease 2019: SARS-CoV-2, severe acute respiratory syndrome coronavirus 2.

FIG. 14. Cross-reactive binding of antibodies against other CoVs in 40 COVID-19 convalescent patients compared to 20 naïve controls. CoV, coronavirus; COVID-19, coronavirus disease 2019.

FIG. 15. Cross-reactive binding of antibodies in 40 COVID-19 convalescent patients compared to 20 naïve controls in protein motifs in other CoVs aligned to SARS-CoV-2. CoV, coronavirus; COVID-19, coronavirus disease 2019; SARS-CoV-2, severe acute respiratory syndrome coronavirus 2

FIG. 16. Supporting information tables (3 tables) show data relevant to the Methods for this study. Table A contains the proteins represented on the array, including the GenBank accession numbers and the number of replicates of reach peptide in those proteins. Supporting information Table B contains the characteristics of the 40 COVID-19 convalescent patients and the 20 naïve controls whose sera were used in this study. Supporting information Table C contains the characteristics of the 40 COVID-19 convalescent patients according to hospitalization status. COVID-19, coronavirus disease 2019.

FIG. 17. A schematic depiction of an immunoassay peptide array of the present invention.

FIG. 18. Tables 9A-12C provide the SARS-CoV-2 epitopes that are contemplated as part of the present invention. These epitopes are contiguous peptides in which the average log 2-normalized intensity in COVID-19 patients was at least 2.0-fold greater than that in controls.

### DETAILED DESCRIPTION

The present disclosure describes several novel, sensitive, and specific peptides in the SARS-CoV-2 proteome that can be used for many different methods and to build kits and assays. For example, the present disclosure describes several peptides in the SARS-CoV-2 proteome that can differentiate people who are currently infected or were previously infected with SARS-CoV-2, people who received the vaccine only (and no prior infection) or people who have never been infected with this virus or vaccinated. The peptides of the present invention may also be used to produce diagnostic tests for SARS-CoV-2 infection or to produce prophylactic and/or therapeutic vaccines that elicit a protective immune response against SARS-CoV-2. The present invention provides peptides (e.g. epitopes or peptides comprising all or part of epitopes) that generate an immune response in a subject against SARS-CoV-2. The inventors found that SARS-CoV-2 infection induces antibodies against peptides found in both structural and non-structural proteins (see Example 1). Specifically, the inventors identified peptides comprising epitopes within the SARS-CoV-2 membrane protein, spike protein, nucleocapsid protein, and several nonstructural proteins. The inventors also discovered that more severely ill SARS-CoV-2 patients exhibit higher-magnitude antibody responses against certain epitopes in SARS-CoV-2. Further, they demonstrated that some antibodies produced against SARS-CoV-2 are cross-reactive with other similar and dissimilar coronaviruses, including “common cold” coronaviruses, other deadly human pathogens, and close relatives of SARS-CoV-2 that have not yet jumped into humans.

Thus, in some embodiments, the present invention provides diagnostic assays, kits, and methods of detecting the presence of antibodies against SARS-CoV-2 in a subject. The presence of the antibodies allows for the identification of subjects who have had a prior SARS-CoV-2 infection. Additionally, methods of screening convalescent sera are provided, which would allow for testing and treatment of subjects having a SARS-CoV-2 infection. Further, the present invention provides compositions and vaccines for producing an antibody response in a subject against SARS-CoV-2.

In one embodiment, peptides identified in the M protein can be used as a diagnostic to distinguish people that have had a prior infection from vaccinated people. The M protein peptides are superior to peptides used currently in diagnostics (current methods use nucleocapsid peptides or proteins) as antibody titers to nucleocapsid are reduced over time, while antibody titers to M protein are detectable at higher levels for longer periods of time, allowing for better diagnostic tools. As described in Example 2, antibodies that bind to peptides of the M protein are detectable over a year after initial infection while titers to nucleocapsid are reduced, provide an advantage to these peptides as diagnostic tools.

In another embodiment, diagnostic assays, kits and methods of detecting and distinguishing a subject that has had a previous SARS-CoV-2 infection or have been vaccinated against SARS-Cov-2 can be detected or distinguished using the peptides of the present invention.

In another embodiment, the epitopes described herein can be used to immunize an animal or human and produce antibodies reactive against these epitopes (including, for example, monoclonal antibodies, chimeric antibodies, humanized antibodies, etc.). These isolated antibodies against epitopes described herein can be used individually or in combination for lateral flow assay or other methods of detecting antigen binding to antibodies, i.e. photon, laser, etc. In some embodiments, these antibodies can further be used for therapy. Using the peptides that were found to be cross-reactive with other coronaviruses, the assays, kits, methods, compositions, and vaccines disclosed herein may be adapted for use with other closely related viruses (e.g., MERS, SARS-CoV), including those from bat and pangolin coronaviruses, as well as pre-emergent coronaviruses (e.g., SARS-CoV-3, SARS-CoV-4).

As described in the Examples, the inventors characterized antibody binding to the full SARS-CoV-2 proteome as well as to the proteome of 6 other human coronaviruses, 21 bat coronaviruses, and pangolin coronaviruses using an ultra-dense peptide array. The reference proteomes of these viruses were tiled on the array as overlapping 16-mer peptides with 1 amino acid offsets. Using secondary antibodies that detect bound IgG with convalescent serum from the UW COVID-19 biobank, the inventors assayed 40 PCR-confirmed positive samples and 20 control samples that were collected before the onset of the pandemic. The results were analyzed to identify peptides that were present at significantly different levels in SARS-CoV-2 infected and uninfected patients.

Peptides that exhibited a 2-fold or greater difference between these patient groups are presented in Tables 1 and 2. Additionally Table 9A (FIG. 19) shows peptides with a p value less than 0.01; Table 9B (FIG. 19) shows peptides with a p value less than 0.1) identified by the methods described herein, and the top 10 peptides are presented in Table 2 which are derived from the peptides of Table 10. Further, peptides that showed a statistically significant difference between severely ill hospitalized patients and the mildly ill, non-hospitalized patients are presented in Tables 11A-11B (Table 11A shows the top 10 peptides; Table 11B shows peptides with a p value less than 0.01). These peptides may be useful to stratify or anticipate disease severity during testing and diagnosis. Thus, combinations of these peptides can be used for kits, assays and methods described herein.

As described in Example 1, the highest magnitude binding of anti-SARS-CoV-2 antibodies from human sera occurred for an epitope in the N-terminus of M protein, with high specificity and sensitivity. Antibodies produced after infection with SARS-CoV-2 reacted with epitopes throughout the proteomes of other human and nonhuman CoVs, recognizing homologous regions across all CoVs. Thus, M epitopes are highly relevant to diagnostic and potentially to vaccine design. SARS-CoV anti-M antibodies can synergize with anti-S and anti-N antibodies for improved neutralization, and M has been used in protective SARS-CoV and MERS-CoV vaccines. Notably, some of the highest binding we observed in the S protein occurred at the base of the extracellular portion of the protein, which would be the site of the putative interaction between SARS-CoV-2 S and M.

The ACE2 binding site and the RBD in general are not as reactive, by these methods, as expected, suggesting that other, less-investigated epitopes may be playing a larger role in immunity to SARS-CoV-2 than is currently appreciated, which is further bolstered by the correlation of some of this binding with neutralizing titers. Our results, in concert with prior knowledge of anti-SARS-CoV antibodies, provide evidence that epitopes in M, particularly the 1-M-24 epitope (SEQ ID NO:1) as well as other novel epitopes identified are targets in SARS-CoV-2 diagnostics, vaccines, and therapeutics.

Antibodies produced in response to SARS-CoV-2 infection appear to bind peptides representing homologous epitopes throughout the proteomes of other human and nonhuman CoVs. Peptides of the present invention demonstrate broad cross-reactivity in some homologous peptide sequences which can be used in the development of pan-CoV vaccines, especially given that antibodies binding to 807-S-26 and 1140-S-25, which showed potential cross-reactivity across all CoVs and all β-CoVs, respectively, are known to be potently neutralizing. Further, the methods described herein efficiently detect antibody binding to linear epitopes.

Further, more severely ill patients have significantly greater reactivity to certain epitopes in S, M, N, and ORF3a. The 9 epitopes with significantly higher magnitude reactivity in intubated patients may play a role in the overaggressive immune response known to characterize severe COVID-19, suggesting that they may be targets for treatment in or prevention of severe disease. The present extensive profiling of epitope-level resolution antibody reactivity in COVID-19 convalescent patients, confirmed by independent assays, provides new epitopes that could serve as important targets in the development of improved diagnostics, vaccines, and therapeutics against SARS-CoV-2 and dangerous human CoVs that may emerge in the future.

### Peptides, Vaccine Compositions and Methods of Use

As demonstrated in the Examples, SARS-CoV-2 infection induces antibodies against previously unreported epitopes in both structural (i.e., membrane protein and nucleocapsid protein) and non-structural proteins. Thus, the present invention provides peptides (i.e., epitopes) that specifically bind anti-SARS-CoV-2 antibodies. The inventors identified these peptides by profiling antibody binding throughout the proteome of SARS-CoV-2 and other coronaviruses.

In one embodiment, the present invention provides one or more peptides detailed in Tables 1-3, 6 and Tables 9-12, which specifically bind to SARS-CoV-2 antibodies within a sample. Additionally, the present invention provides peptides that have at least 90% sequence identity to the peptides detailed in Tables 1-3, 6, and 9-12. Suitably, the peptides comprise about 10-35 amino acids, preferably about 15-20 amino acids in length. The peptides may be used in the detection methods, immunoassays, methods of producing antibodies, or vaccine compositions described herein.

The inventors observed the most sensitive and specific antibody binding response with the SARS-CoV-2 membrane protein (see e.g., peptides in Table 2, e.g., SEQ ID NO:1-16). For example, one membrane protein epitope ITVEELKKLLEQWNLV (SEQ ID NO: 1), contained within peptide MADSNGTITVEELKKLLEQWNLVI (SEQ ID NO:17)), specifically, exhibited greater reactivity in infected patients than in controls, and this peptide could be used to discriminate infected patients from controls with a specificity and sensitivity of 100%. Further, this peptide, SEQ ID NO:1, is able to discern patients that were vaccinated from patients that had had a prior natural SARS-Cov-2 infection. This M protein peptide can be used in combination with a spike protein or peptide to distinguish between vaccinated patients and patients that have had a SARS-Cov-2 infection and patients that had neither vaccine nor infection. Given that all the vaccines available to date are based on the spike protein, vaccinated individuals do not have reactivity to membrane protein (or other SARS-CoV-2 proteins) but do have reactivity to spike protein. Individuals who had SARS-CoV-2 infection before or after vaccination will have reactivity to spike protein or peptides along with other SARS-CoV-2 proteins including M protein and nucleocapsid. The M protein peptide described herein is superior to the current methods of detecting prior infection that rely on nucleocapsid. As demonstrated in Example 2, antibody reactivity to nucleocapsid protein declines over time after infection, while antibody reactivity to our M protein peptide (e.g. SEQ ID NO:1) remains high and detectable, even a year after infection (see, Example 2). As such, the peptides comprising SEQ ID NO:1 may be used as a specific and sensitive diagnostic tool to detect prior SARS-CoV-2 infection.

In one embodiment, a method of distinguishing past SARS-CoV-2 infection vs vaccine or naïve infection is provided. The method comprises contacting a sample from an individual with an M protein peptide selected from Table 1 or 2 (for example, a peptide comprising SEQ ID NO:1), and detecting the presence of antibodies that bind the M protein peptide in the sample. The presence of antibodies to the M protein identifies a person having had a SARS-CoV-2 infection from subject only vaccinated (who would only have anti-spike antibodies 0 or who are naïve to both vaccine and SARS-CoV-2 infection (no SARS-CoV-2 antibodies).

In another embodiment, a method of distinguishing past SARS-CoV-2 infection and vaccinated subjects from naïve subjects is provided. The method comprises contacting a sample from an individual with a spike protein peptide selected from Table 1 or 2 and detecting the presence of antibodies that bind to the spike peptide in the sample. The presence of antibodies to spike protein identifies an individual that has had a prior infection or who has been immunized with a vaccine.

In a further embodiment, a method of distinguishing a person with past SARS-CoV-2 infection, a vaccinated person, or a person who has neither been vaccinated nor infected is contemplated. The method comprises contacting a sample from an individual with an M protein peptide and with a spike protein peptide from Table 1 or 2, and detecting the presence of antibodies to the M peptide, the spike peptide, both the M and spike protein or neither. For example, a sample having antibodies to spike peptide but not membrane peptide is a vaccinated individual (spike positive, M negative). A sample having spike peptide antibodies and M protein antibodies had a past SARS-CoV-2 infection (spike positive, M positive). A sample from a subject that was not vaccinated and did not have a prior infection will have no antibodies to spike peptide or membrane (M) peptide (spike negative, M negative).

The methods, assays and kits described herein can be used for any of the above embodiments. Further, two 16-mer peptide sequences found within this 24-mer membrane protein epitope could also discriminate infected from control with a specificity and sensitivity of 100% (See Examples and Table 2). Other epitopes within the membrane protein were also found to be specific to SARS-CoV-2 infection, but were not equally sensitive markers of infection.

As used herein, the terms “proteins,” “peptides” and “polypeptides” are used interchangeably to designate a series of amino acid residues connected to the other by peptide bonds between the alpha-amino and carboxy groups of adjacent residues. “Protein” and “polypeptide” are often used in reference to relatively large polypeptides, whereas the term “peptide” is often used in reference to small polypeptides, but usage of these terms in the art overlaps. Proteins may include modified amino acids (e.g., phosphorylated, glycated, glycosylated, etc.) and amino acid analogs. The peptides described herein that specifically bind to part of a SARS-CoV-2 protein are about 10 to 35 amino acids in length, more preferably about 15-20 amino acids in length, for example, 10, 11, 12, 13, 14, 15, 16, 17, 18, 19, 20, 21, 22, 23, 24, 25, 26, 27, 28, 29, 30, 31, 32, 33, 34, or 35 amino acids. In one embodiment, the peptides are 16 amino acids in length.

In some embodiments, the peptide or plurality of peptides used with the present invention do not contain a tag and are detected by methods such as photon wavelengths to detect binding to peptides or antibodies, as known in the art.

In some embodiments, the peptide or plurality of peptides used with the present invention further comprise a tag, preferably an exogenous tag or agent. The term “tag” or “agent,” as used herein, includes any useful moiety that allows for the purification, identification, or detection of the peptide(s) of the present invention. Any tag or agent that does not interfere with the ability of the peptide or peptides to bind to the antibodies within the sample may be used with the present invention. Suitable tags are known in the art and include, but are not limited to, affinity or epitope tags (e.g., cMyc, HIS, FLAG, VS-tag, HA-tag, NE-tag, S-tag, Ty tag, universal molecular identifier, magnetic beads, etc.) and florescent tags (e.g., RFP, GFP, etc.). Epitope tags are commonly used as a “purification tag”, i.e. a tag that facilitates isolation of the polypeptide from other non-specific proteins and peptides. For instance, the inventors included a 6X His tag in the peptides to allow them to be purified by nickel affinity chromatography using standard methods known in the art. In some embodiments, the epitope peptide and the tag are encoded in one nucleic acid sequence and translated concurrently. In some embodiments, the tag is cleavable and can be removed once the peptide is expressed and purified. The peptides may be linked directly, linked indirectly, or conjugated to the tag or agent. As used herein, the term “conjugate” refers to the joining of two entities by covalent bonds. The entities may be covalently bonded directly or through linking groups using standard synthetic coupling procedures. For example, two polypeptides may be linked together by simultaneous polypeptide expression, forming a fusion or chimeric protein. One or more amino acids may be inserted into the polypeptide to serve as a linking group (i.e., via incorporation of corresponding nucleic acid sequences into the vector). Other contemplated linking groups include polyethylene glycols or hydrocarbons terminally substituted with amino or carboxylic acid groups to allow for amide coupling with polypeptides having amino acids side chains with carboxylic acid or amino groups, respectively. Alternatively, the amino and carboxylic acid groups can be substituted with other binding partners, such as an azide and an alkyne groups, which undergo copper catalyzed formation of triazoles.

In some embodiments, the peptides comprise multiple tags. For example, a second tag may be included to allow for easy capture of the peptides. Further tags contemplated include, for example, biotin (e.g., via a cysteine or lysine residue), a lipid molecule (e.g., via a cysteine residue), or a carrier protein or peptide. Attachment to tags, such as biotin, can be useful for associating the peptide with ligand receptors, such as avidin, streptavidin, polymeric streptavidin (see, e.g., US 2010/0081125 and US 2010/0267166, both of which are herein incorporated by reference), or neutravidin. Avidin, streptavidin, polymeric streptavidin, or neutravidin, in turn, can be linked to a signaling moiety (e.g., an enzyme, such as horse radish peroxidase (HRP) or alkaline phosphatase (ALP) or β-galactosidase (β-GAL) or other moiety that can be visualized, such as a metallic nanomaterial such as nanoparticle, nanoplate, or nanoshell (e.g., colloidal gold), a fluorescent moiety, or a quantum dot) or a solid substrate (e.g., an Immobilon™ or nitrocellulose membrane or Porex® membrane). Alternatively, the peptides of the invention can be fused or linked to a ligand receptor, such as avidin, streptavidin, polymeric streptavidin, or neutravidin, thereby facilitating the association of the peptides with the corresponding ligand, such as biotin and any moiety (e.g., signaling moiety) or solid substrate attached thereto. Examples of other ligand-receptor pairs are well-known in the art and can similarly be used.

In some embodiments, the one or more peptides may be fused at its N-terminus or C-terminus to another suitable peptide. Two or more copies of a peptide of the invention may be joined to one another, alone or in combination with one or more additional peptides to increase the ability of the peptides to elicit an immune response. Combinations of fused and unfused peptides or polypeptides can be used.

In another aspect, the present disclosure provides a vaccine composition comprising an adjuvant and one or more peptides selected from Tables 1 or Table 9A or 9B or a peptide having at least 90% sequence similarity to one or more epitope peptides in Tables 1 or Table 9A or 9B. In another aspect, the present disclosure provides a vaccine composition comprising an adjuvant and one or more peptides that is a 16-mer or longer peptide of the M protein selected from Tables 1 or Table 9A or 9B or a peptide having at least 90% sequence similarity to one or more epitope peptides that is 16-mer or longer peptide in a Tables 1 or Table 9A or 9B. In another aspect, the present disclosure provides a vaccine composition comprising an adjuvant and one or more peptides that is a 18-mer or longer peptide of the M protein selected from Tables 1 or Table 9A or 9B or a peptide having at least 90% sequence similarity to one or more epitope peptides that is 18-mer or longer peptide in a Tables 1 or Table 9A or 9B. For example, the peptides may be a 16-mer, 17-mer, 18-mer, 19-mer, 20-mer, 21-mer, 22-mer, etc.

In another embodiment, the vaccine composition comprises an adjuvant and one or more peptide are selected from Table 2, one or more peptides that are 16 amino acids or longer or a peptide with at least 90% sequence similarity to a 16-mer or longer peptide of Table 2. In a further embodiment, the vaccine compositions comprise an adjuvant and one or more peptides selected from Tables 11A-11B or a peptide with at least 90% sequence similarity to a peptide in Tables 11A-11B, in some embodiments, the peptides are a 16-mer or longer of the peptides of Table 11A-11B, for example, a 16-mer, a 18-mer, a 20-mer, a 24-mer. For example, the peptides may be a 16-mer, 17-mer, 18-mer, 19-mer, 20-mer, 21-mer, 22-mer, etc.

In a further embodiment, the vaccine composition comprises an adjuvant and two or more peptides, three or more peptides, or four or more peptides selected from Tables 1 or Table 9A-9B. In some embodiments, the peptides selected have at least 90% sequence similarity to a peptide in Tables 1 or 9A-9B, alternatively at least 95% sequence similarity, alternatively at least 98% sequence similarity, alternatively at least 99% sequence similarity, or alternatively are the peptide sequences listed in Tables 1 or 9A-9B.

Table 2 provides the top ten peptides found to be highly specific and sensitive epitopes for discriminating between subjects who have and have not been infected with SARS-CoV-2. In some embodiments, the vaccine comprises two or more peptides, three or more peptides, four or more peptides selected from Table 2, or a peptide having at least 90% sequence similarity to a peptide in Table 2. In some embodiments, the peptides selected have at least 90% sequence similarity to a peptide in Table 2, alternatively at least 95% sequence similarity, alternatively at least 98% sequence similarity, alternatively at least 99% sequence similarity, or alternatively are the peptide sequences listed in Table 2.

Table 11A provides the top ten peptides that comprise highly specific and sensitive epitopes for discriminating between severely ill patients and patients with mild symptoms of COVID-19. Table 11B provides all peptides with a p value less than 0.01. Thus, in some embodiments, the vaccine compositions comprise an adjuvant and two or more peptides, three or more peptides, four or more peptides selected from Tables 11A-11B, or a peptide having at least 90% sequence similarity to a peptide in Tables 11A-11B. In some embodiments, the peptides selected have at least 90% sequence similarity to a peptide in Tables 11A-11B, alternatively at least 95% sequence similarity, alternatively at least 98% sequence similarity, alternatively at least 99% sequence similarity to the peptides listed in Tables 11A-11B, or alternatively are the peptide sequences listed in Tables 11A-11B. Again, it is contemplated that the peptides may be 16-mers or longer of the peptides found in Table 11A-11B, for example, the peptides may be a 16-mer, 17-mer, 18-mer, 19-mer, 20-mer, 21-mer, 22-mer, etc.

Tables 12A-12C provides the peptides that were found to be cross-reactive across two or more coronaviruses. Table 12A provides peptides within nucleocapsid proteins, Table 12B provides peptides within membrane proteins, and Table 12C provides peptides within surface proteins. Thus, in a further embodiment, the vaccine composition comprise an adjuvant and at least one peptide selected from Tables 12A-12C a homologous peptide to one of the peptides of Tables 12A-12C, or peptide with at least 90% sequence similarity to a peptide in Tables 12A-12C, and the peptide is cross-reactive with antibodies to at least two species of coronavirus. Without being bound by any theory, the use of one or more peptides from Tables 12A-12C in a vaccine composition may elicit an immune and antibody response that is cross-reactive against two or more different coronaviruses, making vaccines that comprise these peptides more broadly applicable. Again, it is contemplated that the peptides may be 16-mers or longer of the peptides found in Table 12A-12C, for example, the peptides may be a 16-mer, 17-mer, 18-mer, 19-mer, 20-mer, 21-mer, 22-mer, etc.

In another embodiment, the disclosure provides a method of detecting the presence of a coronavirus in a sample, the method comprising: contacting the sample with one or more peptide selected from Tables 12A-12C, a homologous protein to one or more peptide of Tables 12A-12C, or a peptide having at least 90% sequence similarity to a peptide in Tables 12A-12C; and detecting the binding of one or more antibodies from the sample to the one or more peptide by a detection agent, wherein the presence of the one or more antibodies within the sample indicates the presence of the coronavirus. In some embodiments, the coronavirus is selected from the group consisting of MERS, SARS-CoV, and a pre-emergent coronavirus. Again, it is contemplated that the peptides may be 16-mers or longer of the peptides found in Table 12A-12C, for example, the peptides may be a 16-mer, 17-mer, 18-mer, 19-mer, 20-mer, 21-mer, 22-mer, etc.

Suitable adjuvants are known in the art and include, but are not limited to, threonyl muramyl dipeptide (MDP) (Byars et al., 1987), Ribi adjuvant system components (Corixa Corp., Seattle, Wash.) such as the cell wall skeleton (CWS) component, Freund's complete adjuvants, Freund's incomplete adjuvants, bacterial lipopolysaccharide (LPS; e.g., from E. coli), or a combination thereof. A variety of other well-known adjuvants may also be used with the methods and vaccines of the invention, such as aluminum hydroxide, saponin, amorphous aluminum hydroxyphosphate sulfate (AAHS), aluminum hydroxide, aluminum phosphate, potassium aluminum sulfate (Alum), and combinations thereof. Cytokines (gamma.-IFN, GM-CSF, CSF, etc.), lymphokines, and interleukins (IL-1, IL-2, IL-3, IL-4, IL-5, IL-6, IL-7, IL-8. IL-9, IL-10, IL-11, IL-12, IL-13, IL-14, IL-15, IL-16, IL-17, 11-18, 11-19, IL-20, IL-21, and 11-22) have also been used as adjuvants and/or supplements within vaccine compositions and are contemplated to be within the scope of the present invention. For example, one or more different cytokines and/or lymphokines can be included in a composition comprising one or more peptides or a vaccine of the invention. In a preferred embodiment, the adjuvant is an aluminum salt, AS04, MF59, AS01B, CpG 1018, or another adjuvant that is considered to be safe for use in humans by the Centers for Disease Control and Prevention.

In some embodiments, the vaccine compositions comprise an adjuvant and (a) at least one membrane protein peptide from Tables 1 or a peptide having at least 90% sequence similarity to the at least one membrane protein peptide; and (b) at least one surface protein peptide from Tables 1 or a peptide having at least 90% sequence similarity to the at least one surface protein peptide. Not to be bound by any theory, but the combination of one membrane protein peptide and one surface protein peptide is believed to produce a more robust immune response.

In other embodiments, the vaccine compositions comprise an adjuvant and (a) at least one membrane protein peptide from Tables 1 or a peptide having at least 90% sequence similarity to the at least one membrane protein peptide; and (b) at least one nucleocapsid protein peptide from Tables 1 or a peptide having at least 90% sequence similarity to the at least one nucleocapsid protein peptide.

In further embodiments, the vaccine compositions comprise an adjuvant and (a) at least two membrane protein peptides from Tables 1 or a peptide having at least 90% sequence similarity to the at least two membrane protein peptides. In some embodiments, the vaccine composition may comprise a third peptide derived from a surface protein or a nucleocapsid protein from Tables 1 or a peptide having at least 90% sequence similarity to the at least one surface protein peptide or nucleocapsid protein peptide. Other suitable combinations of one or more peptides listed in Tables 1 are contemplated for use in the present vaccine compositions.

In some embodiments, the vaccine compositions comprise an adjuvant and (a) at least one membrane protein peptide from Tables 9A-9B or a peptide having at least 90% sequence similarity to the at least one membrane protein peptide; and (b) at least one surface protein peptide from Tables 9A-9B or a peptide having at least 90% sequence similarity to the at least one surface protein peptide. Not to be bound by any theory, but the combination of one membrane protein peptide and one surface protein peptide is believed to produce a more robust immune response. In other embodiments, the vaccine compositions comprise an adjuvant and (a) at least one membrane protein peptide from Tables 9A-9B or a peptide having at least 90% sequence similarity to the at least one membrane protein peptide; and (b) at least one nucleocapsid protein peptide from Tables 9A-9B or a peptide having at least 90% sequence similarity to the at least one nucleocapsid protein peptide. In further embodiments, the vaccine compositions comprise an adjuvant and (a) at least two membrane protein peptides from Tables 9A-9B or a peptide having at least 90% sequence similarity to the at least two membrane protein peptides. In some embodiments, the vaccine composition may comprise a third peptide derived from a surface protein or a nucleocapsid protein from Tables 9A-9B or a peptide having at least 90% sequence similarity to the at least one surface protein peptide or nucleocapsid protein peptide. Other suitable combinations of one or more peptides listed in Tables 9A-9B are contemplated for use in the present vaccine compositions.

The vaccine compositions described herein may further comprise a pharmaceutically acceptable excipient. The pharmaceutically acceptable excipient or carrier should be selected based on the selected route of administration and standard pharmaceutical practice. The composition may be formulated into dosage forms according to standard practices in the field of pharmaceutical preparations (see Alphonso Gennaro, ed., Remington's Pharmaceutical Sciences, 18th Ed., (1990) Mack Publishing Co., Easton, Pa). Suitable dosage forms may comprise, for example, solutions, parenteral solutions, or suspensions. In some embodiments, the composition comprises an isolated and purified peptide described herein combined with a suitable adjuvant for administration. In other embodiments, the composition comprises an isolated and purified vector comprising the nucleic acid sequence(s) encoding one or more peptides described herein.

The vaccine compositions as described herein can be used for eliciting an immune response in a subject against SARS-CoV-2. Suitably, the vaccine compositions are administered in a single dosage, or in conjunction with one or more booster dosages. A suitable method of administration can be readily determined by one skilled in the art.

The present disclosure also provides a polynucleotide construct comprising a heterologous promoter region and a nucleotide sequence encoding at least one peptide selected from Tables 1 or Table 9A-9B or a peptide having at least 90% sequence similarity to one or more peptides in Tables 1 or Table 9A-9B. In another embodiment, the polynucleotide encodes at least one peptide selected from Table 2 or a peptide with at least 90% sequence similarity to a peptide in Table 2. In another embodiment, the polynucleotide encodes at least one peptide selected from Tables IA-1B or a peptide with at least 90% sequence similarity to a peptide in Tables 11A-1B.

The terms “polynucleotide,” “polynucleotide sequence,” “oligonucleotide,” “nucleic acid,” and “nucleic acid sequence” are used interchangeably herein to refer to nucleotide sequences or fragments thereof. The polynucleotides of the present invention encode one or more peptides disclosed herein. In some embodiments, the polynucleotide may encode the one or more peptide and a tag. Suitable peptide tags are known in the art and described herein.

In some embodiments, the polynucleotide construct is a vector. Suitable vectors for use with the present invention comprise a promoter operably connected to a polynucleotide sequence encoding the one or more peptides described herein. The vectors may also comprise appropriate control sequences that allow for translational regulation in a host cell. In some embodiments, the vectors further comprise nucleic acid sequences encoding one or more agents or tags. In some embodiments, the vectors further comprise additional regulatory sequences, such as signal sequences.

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors” (or simply, “vectors”). The term vector encompasses “plasmids”, the most commonly used form of vector. Plasmids are circular double-stranded DNA loops into which additional DNA segments (e.g., those encoding one or more peptides) may be ligated. However, other forms of expression vectors, such as viral vectors (e.g., replication defective retroviruses, adenoviruses and adeno-associated viruses), may also be used with the present invention.

In some embodiments, the vectors of the present invention further comprise heterologous backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide. Many commonly used expression vectors and plasmids contain non-human nucleic acid sequences, including, for example, CMV promoters.

Protein and nucleic acid sequence identities are evaluated using the Basic Local Alignment Search Tool (“BLAST”) which is well known in the art (Karlin and Altschul, 1990, Proc. Natl. Acad Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

“Percentage of sequence identity” or “percent similarity” is determined by comparing two optimally aligned sequences over a comparison window, wherein the portion of the polynucleotide or peptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Substantial identity” or “similar identity” of amino acid sequences for purposes of this invention normally means polypeptide sequence identity of at least 90%. Preferred percent identity of polypeptides can be any integer from 90% to 100%. More preferred embodiments include at least 90%, 91%, 92%, 93%, 94%, 95%, 96%, 97%, 98%, 98.7%, or 99%.

In another aspect, the present disclosure provides host cells comprising the polynucleotide constructs or vectors described herein. Any host cell that allows for expression of the peptides encoded by the polynucleotide constructs or vectors may be used with the present invention. For example, common host cells include bacteria (e.g., E. coli, B. subtilis), yeast (e.g., S. cerevisiae), phage or eukaryotic cell lines. For example, phage immunoprecipitation sequencing (PhIP-seq). Advantageously, insect or mammalian cell lines may be used to provide human-like splicing of mRNA. However, those of skill in the art are aware that many expression systems and cell lines may be used to express the peptides of the present invention, including many that are commercially available.

In a further aspect, the disclosure provides a vaccine composition comprising the polynucleotide construct or vector encoding the one or more peptides described herein. These DNA vaccine compositions may further comprise an adjuvant, as known in the art.

The present invention further provides methods of eliciting an immune response to SARS-CoV-2 in a subject. The methods comprise administering an effective amount of a vaccine composition disclosed herein to the subject to elicit an immune response. Suitably, the elicited immune response is a B-cell antibody.

In another embodiment, embodiment, the peptides described herein can be used in methods to produce antibodies that are specific to the peptides in a subject. In some embodiments, the subject is an animal that can produce an antibody. The method comprises administering (preferably by injection) the epitopes of the present invention (e.g., peptides in Table 1, 2, 6, and 9-12). Suitably, the peptides are administered with an adjuvant or immunostimulatory molecules that stimulate the production of antibodies within the subject. Suitable adjuvants are known in the art some of which are described herein above. Methods of detecting antibodies are known in the art (e.g., ELISA, etc).

The antibodies may be monoclonal antibodies, humanized antibodies or chimeric antibodies. Methods of producing antibodies are known in the art. For example, methods of making monoclonal antibodies are readily understood in the art. Further, monoclonal antibodies isolated may be able to be made into humanized and chimeric antibodies with methods known it the art. The present invention provides a method of generating an antibody that specifically binds to a peptide from SARS-CoV-2 described herein or within the Tables described herein the method comprising: a) introducing a peptide immunogen to a mammal in an amount sufficient to produce an antibody to said peptide; b) recovering the antibody from the mammal; c) purifying said antibody. In further embodiments, monoclonal antibodies can be made from the mammal inoculated with the peptide by methods known in the art and making hybridomas that specifically produce a specific antibody. Hybridomas can be obtained by immunizing a mammal, such as a mouse or a rat with the peptides described herein, cell fusion of the immunized animals spleen cells to myeloma cells of a mammal, such as a mouse or a rat in accordance with the method originally outlined by Kohler and Milstein [see Nature, 256, 495 (1975)], and then culturing the fused cells in a selection medium. The hybridomas produced can be screened for antibodies that specifically bind the peptide used as an immunogen.

The term “antibody” is used in the broadest sense and specifically covers monoclonal antibodies, antibody compositions with polyepitopic specificity, bispecific antibodies, diabodies, and single-chain molecules, as well as antibody fragments (e.g., Fab, F(ab′)2, and Fv), so long as they exhibit the desired biological activity. As used herein “immunoglobulin” or “antibody” includes all subclasses of alpha, delta, epsilon, gamma, and mu and also refers to any natural (e.g., IgA and IgM) or synthetic multimers of the four-chain immunoglobulin structure. Antibodies non-covalently, specifically, and reversibly bind an antigen. The term “monoclonal antibody” as used herein refers to an antibody obtained from a population of substantially homogeneous antibodies, i.e., the individual antibodies comprising the population are identical except for possible naturally occurring mutations that can be present in minor amounts. For example, monoclonal antibodies may be produced by a single clone of antibody-producing cells. Unlike polyclonal antibodies, monoclonal antibodies are monospecific (e.g., specific for a single epitope of a single antigen). The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method first described by Kohler et al., Nature, 256: 495, 1975, or can be made by recombinant DNA methods. The “monoclonal antibodies” can also be isolated from phage antibody libraries using the techniques known in the art, for example, as described in Marks et al., J. MoI. Biol, 222: 581-597, 1991. As used herein “chimerized” refers to an immunoglobulin, wherein the heavy and light chain variable regions are not of human origin and wherein the constant regions of the heavy and light chains are of human origin. “Humanized” refers to an immunoglobulin such as an antibody, wherein the amino acids directly involved in antigen binding, the complementarity determining regions (CDR), of the heavy and light chains are not of human origin, while the rest of the immunoglobulin molecule, the framework regions of the variable heavy and light chains and the constant regions of the heavy and light chains, are of human origin.

The terms “effective amount” or “therapeutically effective amount” refer to an amount sufficient to effect beneficial or desirable biological and/or clinical results. For example, therapeutically effective amounts of the peptides of the present invention may be combined with a pharmaceutically acceptable carrier and adjuvant to form a vaccine composition. The vaccine composition can be administered in any of the art-recognized modes. The doses, methods of administration, and suitable pharmaceutically acceptable carriers, diluents, and excipients for use with such methods can readily be determined by a skilled artisan, but will depend on the particular circumstances at hand.

Appropriate dosages may be determined, for example, by extrapolation from animal studies or in clinical trials taking into account body weight of the patient, absorption rate, half-life, disease severity and the like. Suitable booster schedules may be determined by a skilled artisan. For example, the compositions may be given once, twice or yearly, or may be given in a series of booster schedule, for example, once a month, every other month, every 4 months, every 6 months, once a year, once every two years, and any range of time in between.

As used herein, “subject” or “patient” refers to both mammals and non-mammals. “Mammals” include any member of the class Mammalia, such as humans, non-human primates (e.g., chimpanzees, other apes and monkey species), farm animals (e.g., cattle, horses, sheep, goats, and swine), domestic animals (e.g., rabbits, dogs, and cats), and laboratory animals (e.g., rats, mice, and guinea pigs). The term “subject” does not denote a particular age or sex. In one embodiment, the subject is a human.

As used herein, the terms “administering” and “administration” refer to any method of providing a pharmaceutical preparation or composition to a subject comprising the one or more peptides described herein. Such methods are well known to those skilled in the art and include, but are not limited to, transdermal administration, administration by inhalation, nasal administration, and parenteral administration, including injectable such as intramuscular administration, intradermal administration, and subcutaneous administration.

To aid in administration, vaccines may be mixed with a suitable carrier or diluent such as water, oil (e.g., a vegetable oil), ethanol, saline solution (e.g., phosphate buffer saline or saline), aqueous dextrose (glucose) and related sugar solutions, glycerol, or a glycol such as propylene glycol or polyethylene glycol. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

### Diagnostic Immunoassay, Kits and Methods of Use

The present disclosure provides diagnostic kits, assays and methods that are able to detect the presence of antibodies that a subject has produced in response to SARS-CoV-2 infection, allowing for detection of a past SARS-CoV-2 infection in a subject or methods of distinguishing from a vaccinated individual, an individual that has had SARS-CoV-2 infection or who has remained uninfected and unvaccinated. Specifically, in one embodiment, the kits, assays and methods may be used to identify people who have an active infection and have mounted an immune response to the virus or to identify people who have been infected (knowingly or unknowingly) and have recovered, including those who were asymptomatic. The assays and methods are both sensitive and specific to SARS-CoV-2 infection. In another embodiment, the kits, assays and methods can be used to detect between a vaccinated individual, an individual that had or has an active SARS-CoV-2 infection or an individual who has remained uninfected and unvaccinated.

In one embodiment, the assay and methods provided comprise one or more peptides to SARS-Cov-2 as described herein (e.g., Table 1 or Table 2, or in Tables 6, 9-12), preferably wherein the peptides are linked or adhered to a solid or semi-solid support. A secondary agent (e.g., detection agent) is used to detect antibodies from the sample that specifically bind to the peptides in the assay. For example, the detection agent may be an anti-IgG antibody that specifically binds to antibody constant region (e.g., human anti-IgG agent). In another embodiment, antibodies produced from the peptides described herein can be used to detect active infection, wherein the antibodies can bind to the virus, for example using lateral flow assay, in a sample from a subject. In other words, instead of the peptides described herein used as the capture agent, the capture agent would be antibodies that have been developed that specifically bind to a peptide described herein.

The term “sensitivity” or “sensitive” refers to the ability of an assay or method to identify samples containing antibodies to SARS-CoV-2 (true positive rate), and the “specificity,” refers to their ability to identify samples without antibodies to SARS-CoV-2 (true negative rate). The sensitivity of the assays and methods was determined based on their ability to detect antibodies in blood samples from patients who have been confirmed to have COVID-19 using a nucleic acid amplification test (i.e., PCR).

In one aspect, the present disclosure provides methods of detecting the presence of antibodies generated against SARS-CoV-2 in a sample. The methods comprise contacting the sample with one or more peptides selected from Tables 1 or Table 1 or a peptide having at least 90% sequence similarity to a peptide in Tables 1 or Table 2; and detecting the binding of one or more antibodies from the sample to the one or more peptide by a detection agent. In these methods, the presence of the one or more antibodies within the sample indicates the presence of anti-SARS-CoV-2 antibodies. In some embodiments, the contacting step uses two or more peptides, three or more peptides, four or more peptides selected from Tables 1 or Table 2 or a peptide having at least 90% sequence similarity to a peptide in Tables 1 or Table 2. In some embodiments, the peptides selected have at least 90% sequence similarity to a peptide in Tables 1 or Table 2, alternatively at least 95% sequence similarity, alternatively at least 98% sequence similarity, alternatively at least 99% sequence similarity to the peptides listed in Tables 1 or Table 2, or alternatively are the peptide sequences listed in Tables 1 or Table 2. One skilled in the art would understand that peptides comprising slight variations relative to the disclosed peptide sequences may still provide sufficient binding and specificity as to be used with the methods of the present invention. In some embodiments, the peptides may further comprise a tag, e.g., to facilitate purification, detection, or adhesion to a solid substrate for practice of the present invention.

In one embodiment, the methods comprise contacting the sample with one or more peptides selected from Table 2 or a peptide having at least 90% sequence similarity to a peptide in Table 2; and detecting the binding of one or more antibodies from the sample to the one or more peptide by a detection agent, wherein the presence of the one or more antibodies within the sample indicates current or past infection with SARS-CoV-2. Table 2 provides the top ten peptides found to be highly specific and sensitive to SARS-CoV-2 infection. In some embodiments, the contacting step uses two or more peptides, three or more peptides, four or more peptides selected from Table 2 or a peptide having at least 90% sequence similarity to a peptide in Table 2. In some embodiments, the peptides selected have at least 90% sequence similarity to a peptide in Table 2, alternatively at least 95% sequence similarity, alternatively at least 98% sequence similarity, alternatively at least 99% sequence similarity to the peptides listed in Table 2, or alternatively are the peptide sequences listed in Table 2.

In one aspect, the present disclosure provides methods of detecting the presence of antibodies generated against SARS-CoV-2 in a sample. The methods comprise contacting the sample with one or more peptides selected from Tables 9A-9B or a peptide having at least 90% sequence similarity to a peptide in Tables 9A-9B; and detecting the binding of one or more antibodies from the sample to the one or more peptide by a detection agent. In these methods, the presence of the one or more antibodies within the sample indicates the presence of SARS-CoV-2. In some embodiments, the contacting step uses two or more peptides, three or more peptides, four or more peptides selected from Tables 9A-9B or a peptide having at least 90% sequence similarity to a peptide in Tables 9A-9B. In some embodiments, the peptides selected have at least 90% sequence similarity to a peptide in Tables 9A-9B, alternatively at least 95% sequence similarity, alternatively at least 98% sequence similarity, alternatively at least 99% sequence similarity to the peptides listed in Tables 9A-9B, or alternatively are the peptide sequences listed in Tables 9A-9B. One skilled in the art would understand that peptides comprising slight variations relative to the disclosed peptide sequences may still provide sufficient binding and specificity as to be used with the methods of the present invention. In some embodiments, the peptides may further comprise a tag, e.g., to facilitate purification, detection, or adhesion to a solid substrate for practice of the present invention.

In one embodiment, the methods comprise contacting the sample with one or more peptides selected from Table 10 or a peptide having at least 90% sequence similarity to a peptide in Table 10; and detecting the binding of one or more antibodies from the sample to the one or more peptide by a detection agent, wherein the presence of the one or more antibodies within the sample indicates current or past infection with SARS-CoV-2. Table 10 provides the top ten peptides found to be highly specific and sensitive to SARS-CoV-2 infection. In some embodiments, the contacting step uses two or more peptides, three or more peptides, four or more peptides selected from Table 10 or a peptide having at least 90% sequence similarity to a peptide in Table 10. In some embodiments, the peptides selected have at least 90% sequence similarity to a peptide in Table 10, alternatively at least 95% sequence similarity, alternatively at least 98% sequence similarity, alternatively at least 99% sequence similarity to the peptides listed in Table 10, or alternatively are the peptide sequences listed in Table 10.

In another embodiment, the methods comprise contacting the sample with one or more peptides selected from Tables 11A-11B or a peptide having at least 90% sequence similarity to a peptide in Tables 11A-11B; and detecting the binding of one or more antibodies from the sample to the one or more peptide by a detection agent, wherein the presence of the one or more antibodies within the sample indicates current or past infection with SARS-CoV-2. Table 11A provides the top ten peptides found to be highly specific and sensitive to antibodies found in severely ill patients as opposed to patients with mild symptoms of COVID-19, while Table 11B provides peptides with a p value less than 0.01. In some embodiments, the contacting step uses two or more peptides, three or more peptides, four or more peptides selected from Tables 11A-11B or a peptide having at least 90% sequence similarity to a peptide in Tables 11A-11B. In some embodiments, the peptides selected have at least 90% sequence similarity to a peptide in Tables 11A-11B, alternatively at least 95% sequence similarity, alternatively at least 98% sequence similarity, alternatively at least 99% sequence similarity to the peptides listed in Tables 11A-11B, or alternatively are the peptide sequences listed in Tables 11A-11B.

In a preferred embodiment, the contacting step comprises at least one membrane protein peptide selected from Tables 1-3, 6, 9-11 or a membrane protein peptide having at least 90% sequence similarity to the peptide in Tables 1-3, 6, 9-11. In some embodiments, the at least one peptide comprises at least one membrane protein peptide and at least one surface protein peptide, wherein the peptides are listed in Tables 1-3, 6, 9-11. In another embodiment, the at least one peptide comprises at least one membrane protein peptide and at least one nucleocapsid protein peptide found in Tables 1-3, 6,9-11.

The method of contacting the sample with the one or more peptides of the present invention may be carried out by a number of different methods known to those skilled in the art. For example, in one embodiment, the peptides of the present invention may be attached to a solid or semi-solid support. In another embodiment, the one or more peptides may be incorporated into a lateral flow device (e.g., Abbott's BinaxNOW).

In another embodiment, the method of detecting the presence of SARS-CoV-2 virus in a sample from a subject are provided. The method comprises contacting the sample with an antibody that binds to one or more peptides or epitopes described herein, and detecting the presence of the sample peptide(s) bound to the antibody detects SARS-CoV-2 virus in the sample. The detection of bound peptide or epitopes signifies that the sample has virus proteins within it, and therefore the subject has a SARS-CoV-2 infection.

In another embodiment, the method of contacting the sample with an antibody that binds to one or more epitopes of the present invention may be carried out by a number of different methods known to those skilled in the art. For example, in one embodiment, the antibodies of the present invention may be attached to a solid or semi-solid support. In another embodiment, the one or more peptides may be incorporated into a lateral flow device (e.g., Abbott's BinaxNOW).

In some embodiments, kits or assays comprising one or more of the peptides described herein attached or immobilized to a solid or semi-solid support are contemplated herein. Suitable peptides may be any described herein, for example, those described in Table 1, Table 2, Table 3, Table 6, or any one of Tables 9-12. In one embodiment, a kit comprising a lateral flow immunoassay device comprising one or more of the peptides described herein is contemplated. Different kits or assays comprising different peptides are contemplated depending on the readout that one desires. For example, different peptide may be used for detecting if a subject had a past infection or is vaccinated vs the peptide used to test to distinguish and stratify disease severity during testing and diagnosis.

Methods of attaching or immobilizing peptides on a solid or semi-solid support are known in the art. The attachment can be covalent or non-covalent, and can be facilitated by a moiety associated with the peptide that enables covalent or non-covalent binding, such as a moiety that has a high affinity to a component attached to the carrier, support, or surface. Suitable solid supports include, but are not limited to a bead or plurality of beads (e.g., a colloidal particle, a metallic nanomaterial, a nanoparticle, a nanoplate, a nanoshell, a nanorod, a latex bead, polystyrene, polycarbonate, polyacrylate, PVDF, or PMMA, etc.), a flow path in a lateral flow immunoassay device (e.g., a porous membrane), a flow path in an analytical or centrifugal rotor, a blot (western blot, a slot blot, or dot blot), a tube or a well (e.g., in a plate suitable for an ELISA assay), or solid substrate (plastic, glass, etc.). In some embodiments, the solid support comprises metal, glass, a cellulose-based material (e.g., nitrocellulose), or a polymer (e.g., polystyrene, polyethylene, polypropylene, polyester, nylon, polysulfone, etc). The one or more peptides can be attached to or immobilized on the substrate either prior to or after the addition of a sample during an immunoassay. In one preferred embodiment, the peptides comprise biotin tags and are immobilized to wells that are coated with avidin.

In certain embodiments, the substrate is a bead or plurality of beads, such as a colloidal particle (e.g., a colloidal nanoparticle made from gold, silver, platinum, copper, cadmium, metal composites, other soft metals, core-shell structure particles, or hollow gold nanospheres) or other type of particle (e.g., a magnetic bead or a particle or nanoparticle comprising silica, latex, polystyrene, polycarbonate, polyacrylate, PVDF, or PMMA). Such particles can comprise a label (e.g., a colorimetric, chemiluminescent, quantum dot or fluorescent label) and can be useful for visualizing the location of the peptides during immunoassays. In certain embodiments, a terminal cysteine of a peptide of the invention is used to bind the peptide directly to a metallic nanomaterial or nanostructure.

The term “detection agent,” as used herein, refers to an agent that is able to detect the binding of antibodies from the sample to the peptides of the present invention. For example, one suitable detection agent is a secondary antibody or fragment thereof, preferably an anti-human immunoglobulin antibody or fragment thereof that is capable of binding any human antibodies that specifically bind to the SARS-CoV-2 peptides described herein. The antibodies used with the present invention may be anti-human IgG, anti-human IgM, or anti-human IgA antibodies. For example, typical anti-human IgG antibodies bind to the constant regions of the antibody, allowing for detection of any bound human antibodies. Alternatively, to perform seropositivity testing in primates, an anti-non-human primate (NHP) immunoglobulin antibody may be used as a detection agent. Such antibodies are known and readily available in the art. In an alternative embodiment, protein A or protein G are used to detect any bound antibodies.

Suitably, in some embodiments, the detection agent (e.g., secondary antibody or fragments thereof) comprises a detectable tag. The detectable tag facilitates detection of the binding of antibodies within the sample to the peptides described herein. Suitable detectable tags are known in the art, and include, but are not limited to, for example, a protein tag, an enzymatic tag, a biotin tag, a chemiluminescent tag, and a fluorescent tag. Some examples of tags include biotin, conjugated gold, carbon, or colored latex nanoparticles, fluorescent proteins, magnetic beads, and colored polystyrene beads, among others which are known and readily available in the art.

In one embodiment, the assay and methods provided comprise one or more peptides to SARS-Cov-2 as described herein (e.g., Table 1 or Table 2, or in Tables 6, 9-12), preferably wherein the peptides are linked or adhered to a solid or semi-solid support. A secondary agent (e.g., detection agent) is used to detect antibodies from the sample that specifically bind to the peptides in the assay. For example, the detection agent may be an anti-IgG antibody that specifically binds to antibody constant region (e.g., human anti-IgG agent). For example, in one embodiment the immunoassay is an immunochromatographic membrane assay.

The sample for use in the assay includes biological samples obtained from a subject suspected of having or suspected to have a current or past infection with SARS-CoV-2. Suitable biological samples include, but are not limited to, for example, blood sample, saliva sample, sputum sample, stool sample, cerebrospinal fluid sample, bronchiolar lavage, nasopharyngeal lavage, nasopharyngeal swab, serum sample, or plasma sample. In some embodiments, the sample is convalescent plasma, which can be further used for treatment in a subject in need thereof.

Suitably, the samples are obtained from the subject who is suspected of having had, been exposed to, or having recovered from SARS-CoV-2 infection. Suitably, the sample may have been collected from a subject at least 3-5 days after onset of symptoms, alternatively at least 7 days after onset of symptoms, alternatively at least 12 days after onset of symptoms, alternatively at least 14 days after onset of symptoms, alternatively at least 28 days after onset of symptoms, alternatively at least 35 days after onset of symptoms, or may have been collected at any time between or after said range (e.g., 7 days, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, etc.). Alternatively, the sample may be taken from a subject that is unaware if they have had symptoms of COVID-19 or have been exposed to SARS-CoV-2.

In any of the methods described herein, the detecting step may comprise performing an ELISA assay. Alternatively, the detecting step may comprise performing a lateral flow immunoassay. In some embodiments, the methods may be performed using specific assay devices, e.g., high throughput devices, such as chemiluminescent microparticle immunoassay (CMIA) (e.g., Abbott's Alinity Immunoassay Systems or Architect immunoassay systems) or a chemiluminescent immunoassay (CLIA) (e.g., Promega's Lumit™ Immunoassay), which are known in the art.

Further, the peptides that showed statistically significant differences in abundance between severely ill hospitalized patients and the mildly ill, non-hospitalized patients (found in Tables 11A-11B) may be useful to distinguish and stratify disease severity during testing and diagnosis.

In a further embodiment, the peptides can be used to distinguish a subject having or having had a SARS-CoV-2 infection, having been vaccinated or being uninfected and unvaccinated. In this embodiment, at least one peptide to the membrane protein as described herein is used, for example, SEQ ID NO:1. Further, in some embodiments, at least one peptide to the spike protein is used as described herein. A subject having only been vaccinated will only have antibodies that bind to the spike protein, while a person who has had a natural SARS-CoV-2 infection will have antibodies to the membrane protein and spike protein, and therefore, subjects will be able to be distinguished from one another.

The methods, kits and assays described herein may also be used to detect if an adequate immune response has been mounted after vaccination, and the results can be used for diagnostic purposes in deciding if a booster vaccine may be necessary to mount an adequate immune response. The methods, kits and assays described herein could be used for this purpose.

In a further embodiment, the methods, kits and assays described herein may be used to screen convalescent serum for suitable antibodies for use in therapeutic treatment of a subject having SARS-CoV-2. Suitably, the method would detect one or more of the peptides described in Table 1 or Table 2, preferably in Table 2, showing that the serum has adequate antibodies to SARS-CoV-2.

### Immunoassay

In another embodiment, the present disclosure provides an immunoassay for detecting antibodies to SARS-CoV-2 in a biological sample. The assay comprises a capture agent comprising one or more peptides selected from Tables 1-3, 9-12 or a peptide with at least 90% similarity to an epitope peptide in Tables 1-3, 9-11 (alternatively at least 95% similarity), and a detection agent capable of binding to the one or more antibodies that bind to the capture agent. In some embodiments, the capture agent is one or more peptides selected from Table 2.

In some embodiments, the one or more peptides are attached to a solid or semi-solid support. In other embodiments, the one or more peptides are within a lateral flow device or an immunochromatographic membrane assay.

Suitable detection agents are described herein and include, for example, an anti-human antibody or fragment thereof that binds to human IgG, IgM, or IgA. The detection agent may have a detectable tag. In other embodiments, the detection agent is tagless and uses photons. Suitable detectable tags are described herein and include, for example, an enzymatic tag, a biotin tag, a chemiluminescent tag and a fluorescent tag.

The capture agent may comprise one or more peptides, two or more peptides, three or more peptides that would ensure accurate, specific, and selective detection of SARS-CoV-2. In some embodiments, the capture agent comprises at least one membrane protein-specific peptide selected from Tables 1-3, 6, 9-11 or a peptide having at least 90% sequence similarity to the membrane-specific epitope in Tables 9A-1B. One suitable detection agent is a secondary antibody or fragment thereof, preferably an anti-human IgG, IgM, or IgA antibody or fragment thereof. In another embodiment, the capture agent may comprise one or more peptides of Table 12, or a peptide having at least 90% sequence similarity to a peptide of Table 12, and may be used as an immunoassay to test for one or more coronaviruses (e.g., a pan coronavirus test).

In some embodiments, the immunoassay is a lateral flow detection system. Lateral flow detection systems are known and understood in the art, for example, as described in Koczula K M, Gallotta A. Lateral flow assays. Essays Biochem. 2016; 60(1):111-120. doi:10.1042/EBC20150012, the contents of which are incorporated by reference in its entirety. In brief, lateral flow device is a simple device that contains paths within the device through different zones in which the sample will flow by capillary action and can bind to the one or more peptides described herein, the detection agent and provide a readable signal. For example, the lateral flow strip may comprise an adsorbent sample pad in which to intake the sample, conjugate release pad, and a detection zone. The read-out may be represented by a line appearing at different intensities that can be assessed by eye or a dedicated reader. Lateral flow devices can contain multiple array channels if more than one antibody is to be assayed.

In some embodiments, the immunoassay is a lateral flow detection system that contains paths within the device through different zones in which the sample will flow by capillary action and can bind to the one or more antibodies that can bind to one or more of the peptides described herein, the detection agent and provide a readable signal. This immunoassay could be used to detect current or new infections. For example, a lateral flow device similar to the above using antibodies that specifically bind the peptides described herein is contemplated (e.g., peptides of Tables 1, 2, 6, 9-12) for the detection of SARS-CoV-2 peptides or proteins within a sample, signifying a virally infected subject.

For example, a suitable lateral flow immunoassay format comprises antibodies to human immunoglobulins labeled with a signal generator or detection agent (e.g., colloidal gold) that is dried and placed on a glass fiber pad (sample application pad or conjugate pad). The one or more peptides of the present invention are immobilized on a membrane, such as nitrocellulose or a PVDF (polyvinylidene fluoride) membrane (e.g., an Immobilon™ membrane). A sample (e.g., blood, serum, etc.) is applied to the sample application pad (or flows through the conjugate pad). The sample dissolves the labeled detection agent, which then binds to all antibodies in the sample. The resulting detection agent-antibody complexes are then transported into the next membrane (PVDF or nitrocellulose), which contains the diagnostic peptide, by capillary action. If antibodies against the one or more peptides are present in the sample, they bind the one or more peptides striped on the membrane, thereby generating a signal (e.g., a band that can be seen or visualized). An additional antibody specific to the labeled antibody or a second labeled antibody can be used to produce a control signal.

In an alternative example, the lateral flow immunoassay comprises the one or more peptides being conjugated to a detectable tag (e.g., biotin) and complexed with labeled ligand receptor (e.g., streptavidin-colloidal gold). The tagged peptide complexes can be placed on the sample application pad or conjugate pad. Anti-human IgG/IgM antibodies or other peptides of the invention are immobilized on a membrane, such as nitrocellulose or PVDF, or Porex® membrane at a test site (e.g., a test line). When sample is added to the sample application pad, antibodies in the sample react with the tagged peptide complexes such that antibodies that bind to peptides of the invention become indirectly labeled. The antibodies in the sample are then transported into the next membrane (PVDF, Porex® membrane, or nitrocellulose), which contains the diagnostic peptide, by capillary action and bind to the immobilized anti-human IgG/IgM/IgA antibodies (or protein A, protein G, protein A/G fusion proteins, protein L, or combinations thereof) or immobilized peptides of the invention. If any of the sample antibodies are bound to the labeled peptides of the invention, the label associated with the peptides can be seen or visualized at the test site.

Other lateral flow device designs are understood in the art and contemplated as part of the present invention.

In other embodiments, the immunoassay is an enzyme-linked immunosorbent assay (ELISA). ELISA is a plate-based assay technique wherein the one or more peptides is immobilized on a solid surface (microplate), exposed to the sample for binding of antibodies within the sample, and then incubated with a detection agent, i.e., anti-human secondary antibody that is typically linked to a reporter enzyme. Detection is accomplished by measuring the activity of the reporter enzyme via incubation with the appropriate substrate to produce a measurable product. In one embodiment, the one or more peptides of the invention is immobilized on a surface, such as a ninety-six-well ELISA plate or equivalent solid phase that is coated with streptavidin or an equivalent biotin-binding compound, such as avidin or neutravidin, at an optimal concentration in an alkaline coating buffer and incubated at 4° C. overnight. After a suitable number of washes with standard washing buffers, an optimal concentration of a biotinylated form of the one or more peptides is applied to each well. A sample is then added, and incubated for a sufficient length of time to allow specific binding to occur. The well(s) is washed to remove unbound protein and then incubated with an appropriate anti-immunoglobulin antibody (e.g., for human subjects, an anti-human immunoglobulin from another animal, such as dog, mouse, rat, pig, cow, etc.) that is labeled with a detection agent. In one embodiment, the detection agent is an enzyme, for example, horseradish peroxidase (HRP), beta-galactosidase, alkaline phosphatase (ALP), glucose oxidase, O-GAL, etc. Sufficient time is allowed for specific binding to occur, then the well is washed again to remove unbound conjugate, and a suitable substrate for the enzyme is added. Color is allowed to develop and the optical density of the contents of the well is determined visually or instrumentally (measured at an appropriate wave length). Conditions for performing ELISA assays are well-known in the art. Other ELISA designs are well understood by one skilled in the art and contemplated within the present invention.

In further embodiments, the immunoassay is a chemiluminescent microparticle immune assay (CMIA), a chemiluminescent immunoassay (CLIA), or another high throughput assay for detecting an antibody in a sample. These high throughput assays are known and understood in the art.

The present disclosure also contemplates the use of the immunoassay described herein for the detection of SARS-CoV-2 in a biological sample.

As used herein, the term “specifically binds” or “specific binding” refers to the specificity of an antibody such that it preferentially binds to a defined target (e.g., peptides of the present disclosure). An antibody “specifically binds” to a target if it binds with greater affinity, avidity, more readily, and/or with greater duration than it binds to other substances. For example, an antibody that specifically binds to a target may bind to the target with at least about 10%, at least about 20%, at least about 30%, at least about 50%, at least about 60%, at least about 70%, at least about 80%, at least about 90%, or more, greater affinity as compared to binding to other substances; or with at least about two-fold, at least about five-fold, at least about ten-fold, or more of the affinity for binding to other substances. Recognition by an antibody of a particular target in the presence of other potential targets is one characteristic of such binding. Specific binding of the presently contemplated antibodies to particular targets is measured through known methods utilizing the tools provided herein.

### Kits

The present disclosure further provides kits comprising the immunoassay described herein or kits for carrying out the methods described herein. In one embodiment, the kit comprises the immunoassay described herein. In another embodiment, the present disclosure provides kits comprising one or more peptides of the present invention, and may further include a detection agent able to recognize human antibodies that may bind to the one or more peptides. In one embodiment, the detection agent is an anti-human IgG, IgM, or IgA antibody conjugated to a detectable label. In some embodiments, the detectable label is as described herein and may include, for example, an enzyme, a metallic nanomaterial, a fluorophore, or colored latex particle.

In some embodiments, the peptides in the kit are attached to or immobilized on a solid or semi-solid support. In certain embodiments, the solid support is a bead (e.g., a colloidal particle, a metallic nanomaterial such as nanoparticle, nanoplate, nanoshell, a latex bead, etc.), a flow path in a lateral flow immunoassay device, a flow path in an analytical device, a tube or a well (e.g., in a plate or microtiter plate).

In some embodiments, the kits further comprise a population of beads or a plate (e.g., a plate suitable for an ELISA assay). In other embodiments, the kits further comprise a device, such as a lateral flow immunoassay device, a western blot, a dot blot, a slot blot, an electrochemical sensor, an optical sensor, or an opto-electronic sensor. In certain embodiments, the population of beads, the plate, or the device is useful for performing an immunoassay. For example, in certain embodiments, the population of beads, the plate, or the device is useful for detecting formation of an antibody-peptide complex comprising an antibody from a sample and a peptide of the invention. In certain embodiments, a peptide or population of different peptides of the invention is attached to or immobilized on the beads, the plate, or the device.

The kits of the invention may further comprise a set of instructions indicating, for example, how to use the one or more peptides of the invention to detect antibodies to SARS-CoV-2.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1: Landscape of Antibody Binding in SARS-CoV-2 Infection

The search for potential antibody-based diagnostics, vaccines, and therapeutics for pandemic severe acute respiratory syndrome coronavirus 2 (SARS-CoV-2) has focused almost exclusively on the spike (S) and nucleocapsid (N) proteins. Coronavirus membrane (M), ORF3a, and ORF8 proteins are humoral immunogens in other coronaviruses (CoVs) but remain largely uninvestigated for SARS-CoV-2. Here, we use ultradense peptide microarray mapping to show that SARS-CoV-2 infection induces robust antibody responses to epitopes throughout the SARS-CoV-2 proteome, particularly in M, in which one epitope achieved excellent diagnostic accuracy. The peptides specific for SARS-CoV-2 of the present invention were identified as at least 2 contiguous, overlapping peptides in which the average log 2-normalized intensity in the patients was at least 2.0-fold greater than that in the controls and for which t-tests produced adjusted p-values of <0.01. All patients showed IgG antibody binding to proteins in the SARS-CoV-2 proteome (GenBank Accession: NC_045512.2). The inventors observed responses specific to SARS-CoV-2 infection status in both structural and non-structural proteins. Specifically, peptides comprising infection-specific epitopes were identified in the SARS-CoV-2 spike, membrane, nucleocapsid, orf3a, orf6, and orfB proteins. The peptides or epitopes detailed in some embodiments are named by the position in the protein in which they are found, the protein (S-spike, M-membrane, N-nucleocapsid, etc) and the epitope's length. The inventors found the highest fluorescence intensity and the most sensitive and specific responses in antibody binding to the SARS-CoV-2 membrane protein. All patient sera showed consistent responses in the SARS-CoV-2 membrane protein in one epitope located at the N-terminus (specifically ITVEELKKLLEQWNLV (SEQ ID NO:1), found in MADSNGTITVEELKKLLEQWNLVI (SEQ ID NO:17), which was not recognized by any control. Other epitopes in membrane protein were similarly specific to COVID-19 patients.

Patient sera showed specific reactivity in several epitopes in SARS-CoV-2 spike protein, particularly in three epitopes in the S2 region (amino acids 686-1273). The inventors did not detect any linear epitopes in the receptor binding domain (RBD, amino acids 319-541), though one epitope detected (TESNKKFLPFQQFGRDIADTTDA (SEQ ID NO:97) or TESNKKFLPFQQFGRDIADTTDAVRD (SEQ ID NO:23)) is immediately adjacent to the RBD. The inventors also observed sensitive and specific binding in the nucleocapsid protein, while antibody binding throughout SARS-CoV-2 envelope protein was sparse in controls and patients alike. Antibody binding to non-structural proteins, though less sensitive, was specific for SARS-CoV-2 infection. As described above, these peptides can be used in compositions, vaccines, vectors, diagnostics, kits, methods for detecting SARS-CoV-2 infection and methods of inducing an immune response against SARS-CoV-2, as described herein and throughout the Exhibits.

In this Example, the inventors map 79 B cell epitopes throughout the SARS-CoV-2 proteome and demonstrate that antibodies that develop in response to SARS-CoV-2 infection bind homologous peptide sequences in the 6 other known human CoVs. Reactivity against 4 of our top-ranking epitopes by enzyme-linked immunosorbent assay (ELISA) was also confirmed. Illness severity correlated with increased reactivity to 9 SARS-CoV-2 epitopes in S, M, N, and ORF3a in our population. Our results demonstrate previously unknown, highly reactive B cell epitopes throughout the full proteome of SARS-CoV-2 and other CoV proteins.

Results

### SARS-CoV-2-Naïve Controls Show Consistent Binding in “Common Cold” CoVs and Limited Binding in SARS-CoV-2, SARS-CoV, and MERS-CoV

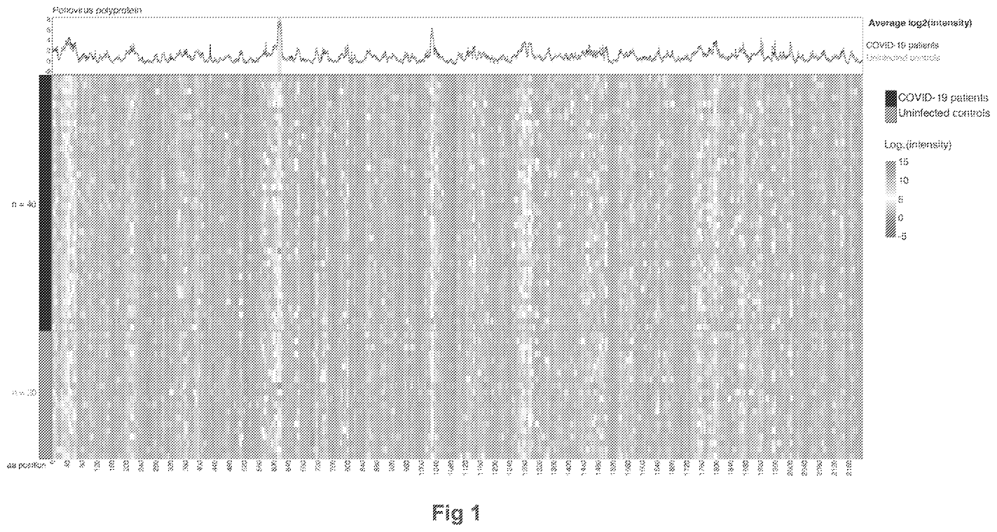

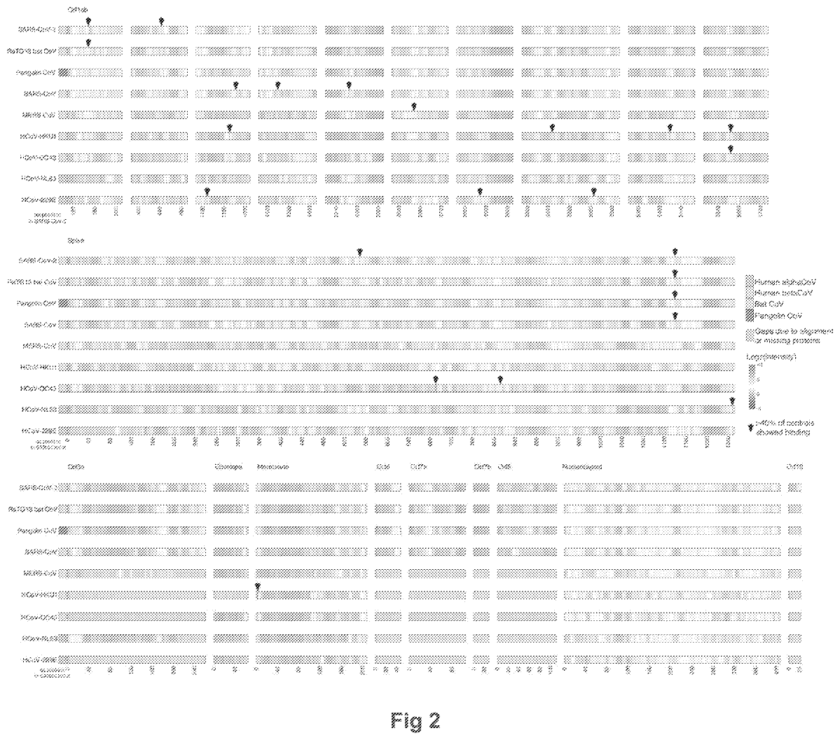

Greater than 90% of adult humans are seropositive for the human “common cold” CoVs (CCCoVs: HCoV-HKU1, HCoV-OC43, HCoV-NL63, and HCoV-229E), but the effect of these preexisting antibodies upon immune responses to SARS-CoV-2 or other CoVs remains uncertain. We measured IgG reactivity in sera from 20 SARS-CoV-2-naïve controls to CoV linear peptides, considering reactivity that was >3.00 standard deviations above the mean for the log₂-quantile normalized array data to be indicative of antibody binding. All sera (SARS-CoV-2-naïve and COVID-19-convalescent) exhibited binding in known epitopes of at least one of the control non-CoV strains (poliovirus vaccine and rhinovirus; FIG. 1), and all were collected in Wisconsin, United States of America, where exposure to SARS-CoV or MERS-CoV was extremely unlikely. We found that at least 1 epitope in structural or accessory proteins showed binding in 100% of controls for HCoV-HKU1, 85% of controls for HCoV-OC43, 65% for HCoV-NL63, and 55% for HCoV-229E (FIG. 2). The apparent cross-reactive binding was observed in 45% of controls for MERS-CoV, 50% for SARS-CoV, and 50% for SARS-CoV-2. We completed neutralization assays on 12 of these control samples and on 18 additional samples from other SARS-CoV-2-naïve controls collected before 2019, and none of these had detectable neutralization activity against SARS-CoV-2 (FIG. 9).

SARS-CoV-2 Infection Induces Antibodies Binding Throughout the Proteome

We aimed to map the full breadth of IgG binding induced by SARS-CoV-2 infection and to rank the identified epitopes in terms of likelihood of immunodominance. We defined epitope recognition as antibody binding to contiguous peptides in which the average log₂-normalized intensity for patients was at least 2-fold greater than for controls with t test statistics yielding adjusted p-values <0.1. We chose these criteria, rather than the 3.00 standard deviation cutoff, in order to ensure that binding detected would be greater than background binding seen in controls (2-fold greater) and to remove regions of binding that were not at least weakly significantly different from controls (adjusted p<0.1). All COVID-19 convalescent patients' sera bound multiple epitopes in SARS-CoV-2, including in 2 patients who did not have detectable neutralizing antibodies in neutralization assays (FIG. 9). Top-ranking epitopes had greater correlations (0.7 and greater) with neutralization titers

TABLE 1
Profiling antibody binding in 40 COVID-19 convalescent patients compared to 20 naive
controls identifies B cell epitopes in SARS-CoV-2 (all data is log₂-normalized)

| | | | | | | | | | | | | SEQ |
|---|---|---|---|---|---|---|---|---|---|---|---|---|
| | | | | | | | | | | | | ID |
| Protein | [symbol] | [symbol] | Sequence | [symbol] | [symbol] | [symbol] | [symbol] | [symbol] | [symbol] | [symbol] | [symbol] | NO: |
| [symbol] | [symbol] | [symbol] | [symbol] | [symbol] | [symbol] | [symbol] | [symbol] | [symbol] | [symbol] | [symbol] | [symbol] | [symbol] |

aa, amino acid; COVID-19, coronavirus disease 2019; M, membrane; N, nucleocapsid: S, spike; SARS-CoV-2, severe acute respiratory syndrome coronavirus 2. [symbol]
indicates data missing or illegible when filed

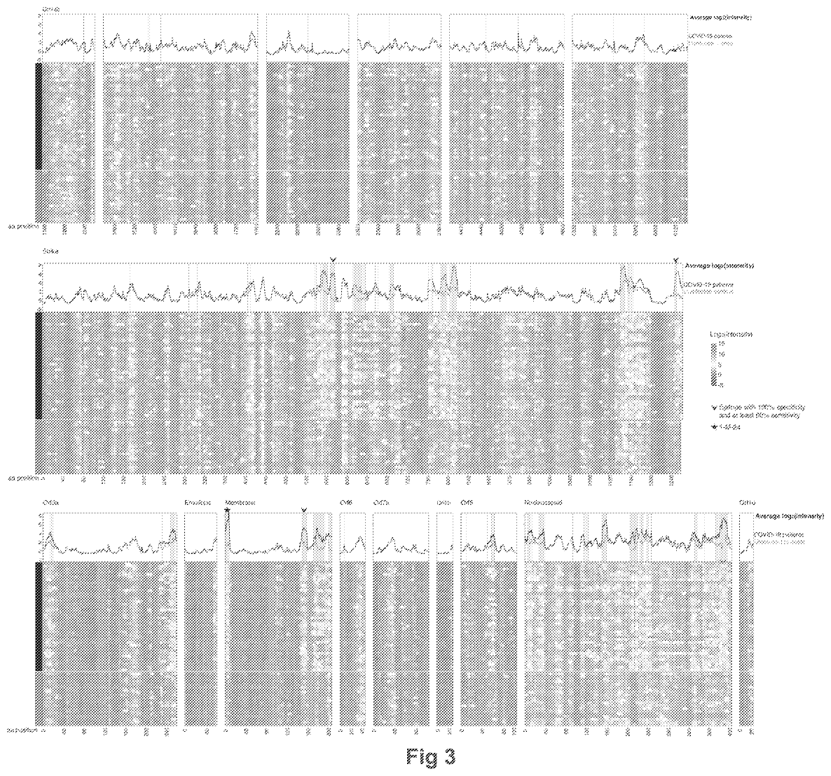

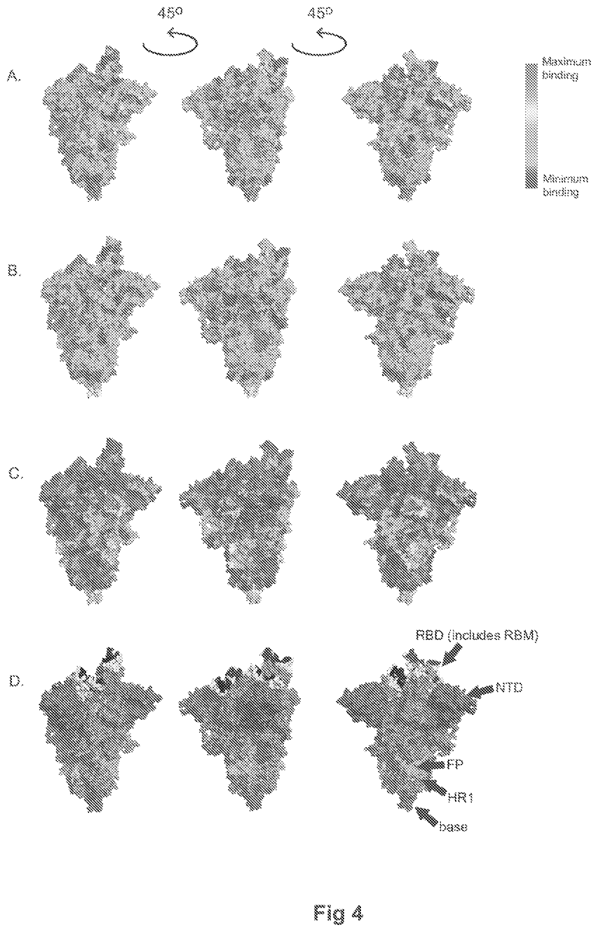

These criteria identified 79 B cell epitopes (FIG. 3, Table 1) in S, M, N, ORFlab, ORF3a, ORF6, and ORF8. We ranked these epitopes by minimum adjusted p-value for any 16-mer in the epitope in order to determine the greatest likelihood of difference from controls as a proxy for likelihood of immunodominance. The highest-ranking epitope occurred in the N-terminus of M (1-M-24). Patient sera showed high magnitude reactivity (up to an average of 6.7 fluorescence intensity units) in other epitopes in S, M, N, and ORF3a, with lower-magnitude reactivity (average of <3.3 fluorescence intensity units) epitopes in other proteins. The epitopes with the greatest reactivity in S were located in the S2 subunit of the protein (residues 686-1273) rather than the S1 subunit (residues 14-685) (FIG. 3). The greatest reactivity in S occurred in the fusion peptide (residues 788-806) and at the base of the extracellular portion of the protein (between the heptad repeat 1 and heptad repeat 2, roughly residues 984-1163) (FIGS. 3 and 4). The highest magnitude antibody binding (red sites in FIG. 4A) on S are below the flexible head region that must be in the “up” position for angiotensin converting enzyme 2 (ACE2) binding to occur. Notably less reactivity occurred in the receptor-binding domain (RBD) (residues 319-541). Four detected epitopes (553-5-26, 624-5-23, 807-S-26, and 1140-S-25) have previously been shown to be potently neutralizing (Poh C M, Carissimo G, Wang B, Amrun S N, Lee C Y, Chee R S, et al. Two linear epitopes on the SARS-CoV-2 spike protein that elicit neutralising antibodies in COVID-19 patients. Nat Commun. 2020; 11:2806. pmid: 32483236; Zhang B Z, Hu Y F, Chen L L, Yau T, Tong Y G, Hu J C, et al. Mining of epitopes on spike protein of SARS-CoV-2 from COVID-19 patients. Cell Res. 2020; 30(8):702-4. pmid: 32612199; Li Y, Lai D Y, Zhang H N, Jiang H W, Tian X, Ma M L, et al. Linear epitopes of SARS-CoV-2 spike protein elicit neutralizing antibodies in COVID-19 patients. Cell Mol Immunol. 2020; 17:1095-7. pmid: 32895485), and all 4 of these were ranked within the top 10 of our 79 epitopes. Forty-two of our detected epitopes (including 1-M-24, 553-S-26, 624-S-23, 807-S-26, and 1140-S-25; Table 1) confirm bioinformatic predictions of antigenicity based on SARS-CoV and MERS-CoV, including each of the 12 top-ranking epitopes.

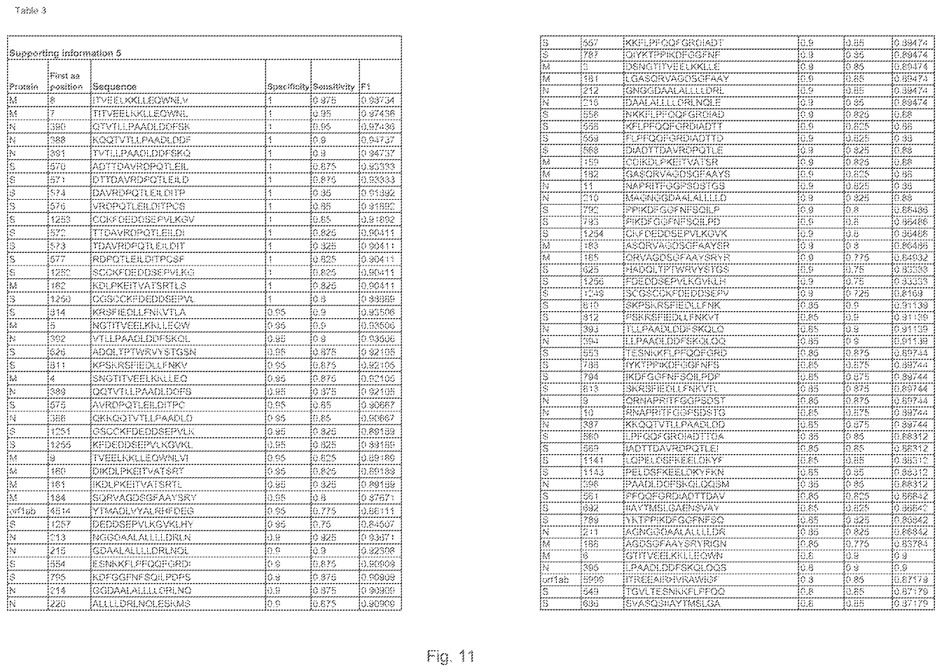

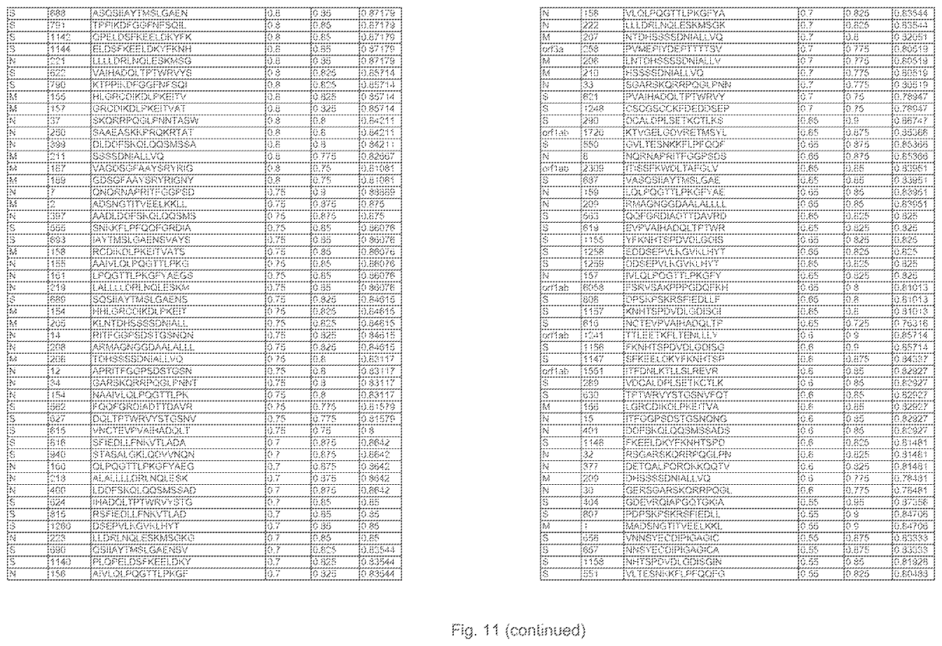

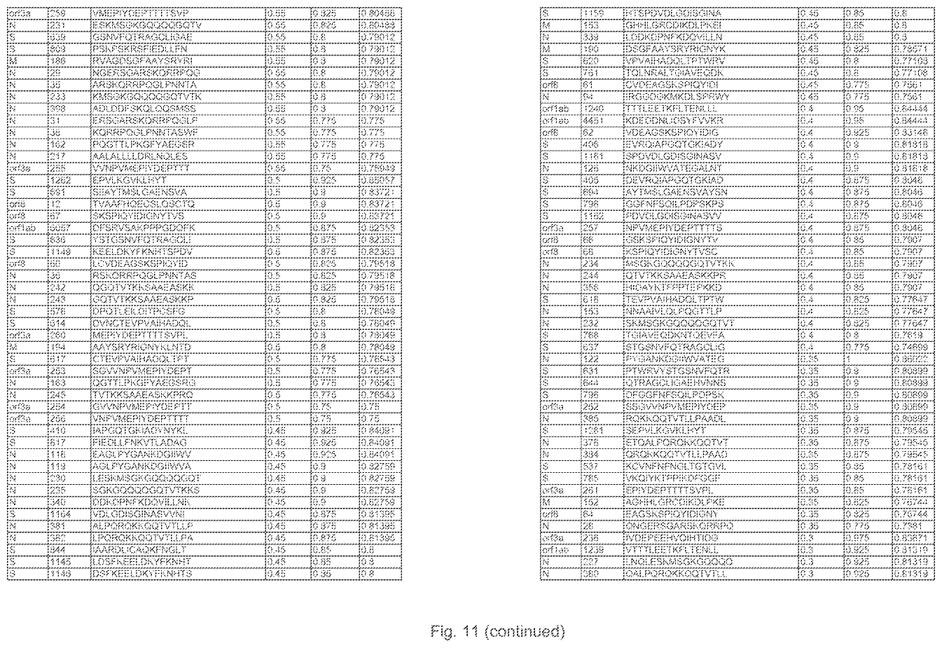

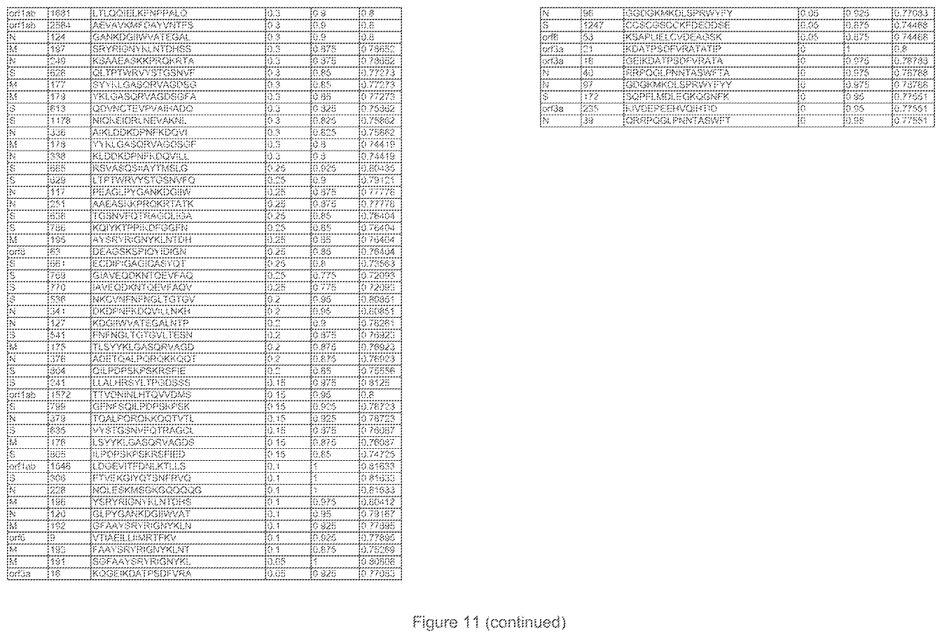

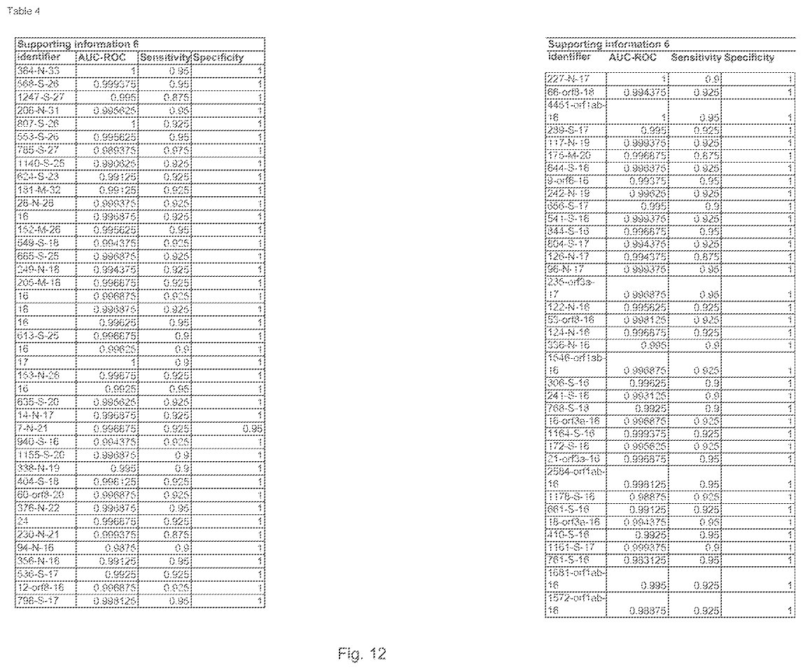

The highest specificity (100%) and sensitivity (98%), determined by linear discriminant analysis leave-one-out cross-validation, for any individual peptide was observed for a 16-mer within the 1-M-24 epitope: ITVEELKKLLEQWNLV (SEQ ID NO:1, FIG. 11 (Table 3)). Fifteen additional individual peptides in M, S, and N had 100% measured specificity and at least 80% sensitivity (Table 2, derived from epitopes found in Table 10A). Combinations of 1-M-24 with 1 of 5 other epitopes (384-N-33, 807-S-26, 6057-ORFlab-17, 227-N-17, and 4451-ORFlab-16) yielded an area under the curve receiver operating characteristic of 1.00 (FIG. 12 (Table 4)) based on linear discriminant analysis leave-one-out-cross-validation.

TABLE 2
Sixteen peptides in the SARS-CoV-2 proteome had 100% specificity and at
least 80% sensitivity for SARS-CoV-2 infection in 40 COVID-19 convalescent
patients compared to 20 naive controls.

| | First aa | | SEQ | Speci- | Sensi- | |
|---|---|---|---|---|---|---|
| Protein | position | Sequence | ID NO: | ficity | tivity | F1 |
| M | 8 | ITVEELKKLLEQWNLV | 1 | 1 | 0.98 | 0.99 |
| M | 7 | TITVEELKKLLEQWNL | 2 | 1 | 0.95 | 0.97 |
| N | 390 | QTVTLLPAADLDDESK | 3 | 1 | 0,95 | 0.97 |
| N | 388 | KQQTVILLPAADLDDF | 4 | 1 | 0.90 | 0.95 |
| N | 391 | TVILLPAADLDDFSKQ | 5 | 1 | 0.90 | 0.95 |
| S | 570 | ADTTDAVRDPQTLEIL | 6 | 1 | 0.88 | 0.93 |
| S | 571 | DTTDAVRDPQTLEILD | 7 | 1 | 0.88 | 0.93 |
| S | 574 | DAVRDPQTLEILDITP | 8 | 1 | 0.85 | 0.92 |
| S | 576 | VRDPQTLEILDITPCS | 9 | 1 | 0.85 | 0.92 |
| S | 1253 | CCKFDEDDSEPVLKGV | 10 | 1 | 0.85 | 0.92 |
| S | 572 | TTDAVRDPQTLEILDI | 11 | 1 | 0.83 | 0.90 |
| S | 573 | TDAVRDPQTLEILDIT | 12 | 1 | 0.83 | 0.90 |
| S | 577 | RDPQTLEILDITPCSF | 13 | 1 | 0.83 | 0.90 |
| S | 1252 | SCCKFDEDDSEPVLKG | 14 | 1 | 0.83 | 0.90 |
| M | 162 | KDLPKEITVATSRTLS | 15 | 1 | 0.83 | 0.90 |
| S | 1250 | CGSCCKFDEDDSEPVL | 16 | 1 | 0.80 | 0.89 |

aa, amino acid; COVID-19, coronavirus disease 2019; M, membrane; N, nucleocapsid; S, spike; SARS-CoV-2, severe acute respiratory syndrome coronavirus 2.

Anti-SARS-CoV-2 Antibodies May Cross-Reactively Bind Peptides in Other CoVs

We determined epitopes bound by anti-SARS-CoV-2 antibodies in non-SARS-CoV-2 CoVs by the same criteria we used to determine epitopes in SARS-CoV-2. Epitopes in these viruses were defined as binding by antibodies in COVID-19 convalescent sera to peptides at an average log₂-normalized intensity at least 2-fold greater than in controls with t test statistics yielding adjusted p-values <0.1. Some of these epitopes were identical sequences with SARS-CoV-2, particularly in the RaTGI3 bat betacoronavirus (0-CoV), the closest known relative of SARS-CoV-2 (96% nucleotide identity), the pangolin CoV (85% nucleotide identity with SARS-CoV-2), and SARS-CoV (78% identity). Cross-reactivity of an antibody is typically determined by evaluating a pure preparation of specific antibodies or by competition assays. However, since our Wisconsin controls are almost certainly naïve to MERS-CoV, SARS-CoV, and bat and pangolin CoVs, we can make predictions about cross-reactivity (as opposed to binding due to sequence identity).

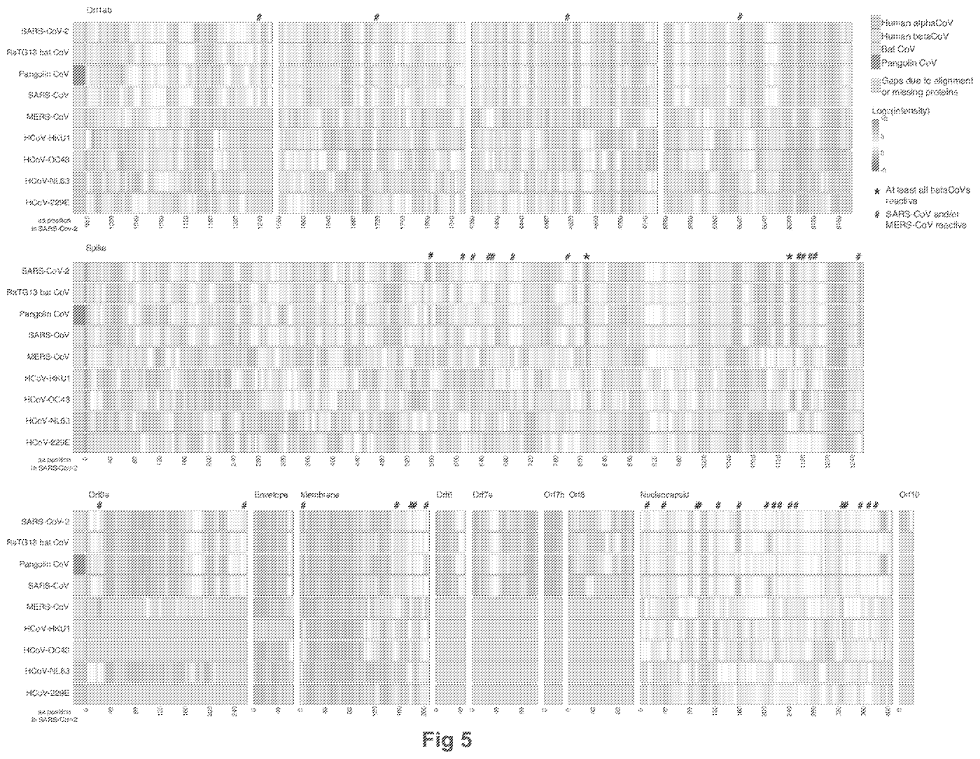

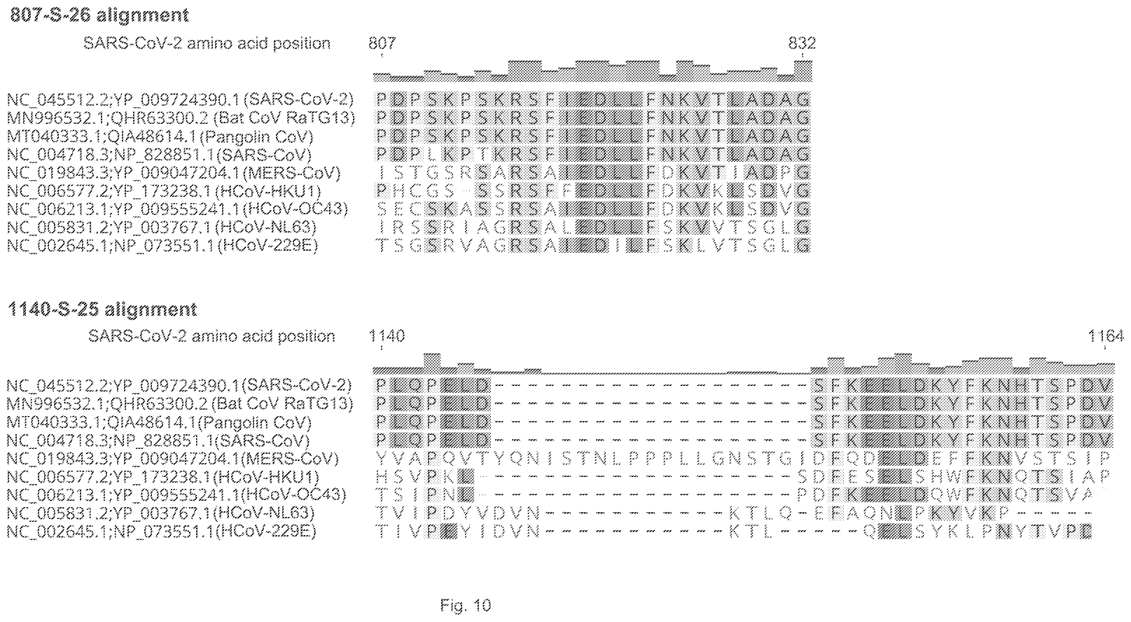

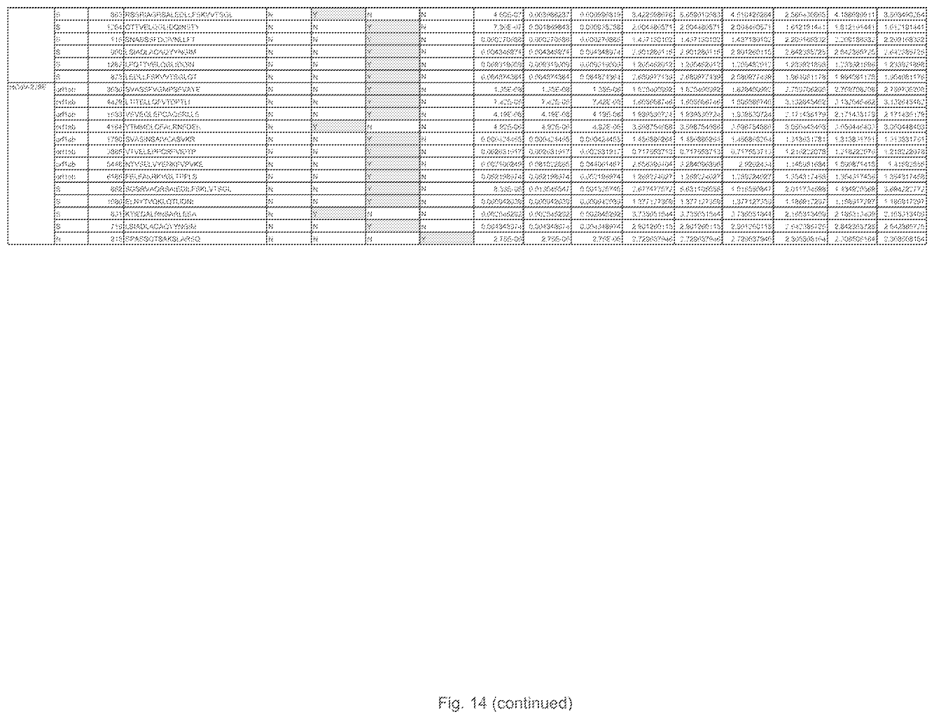

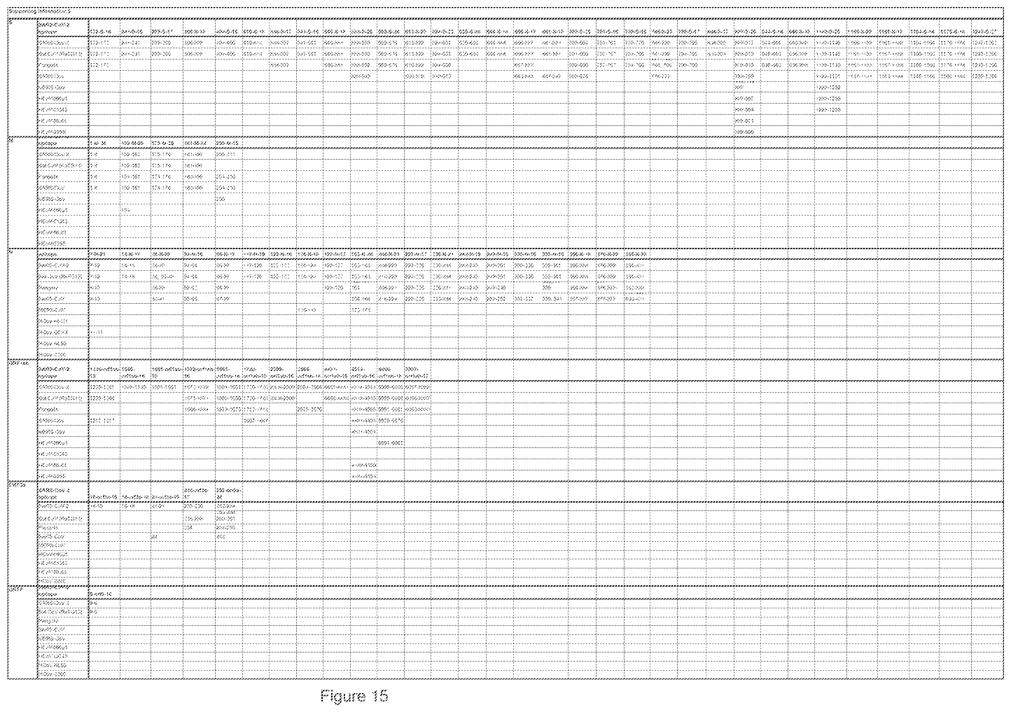

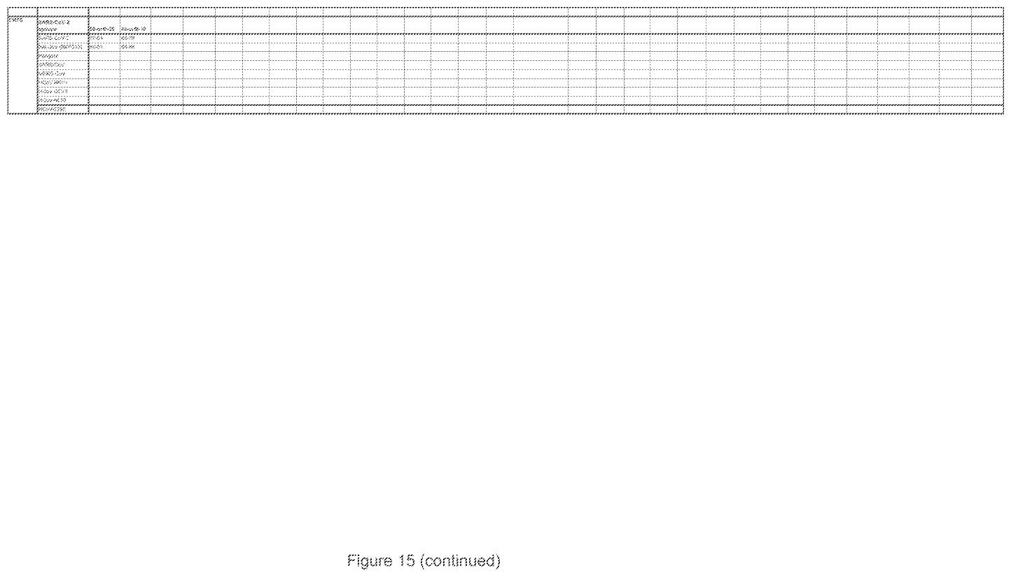

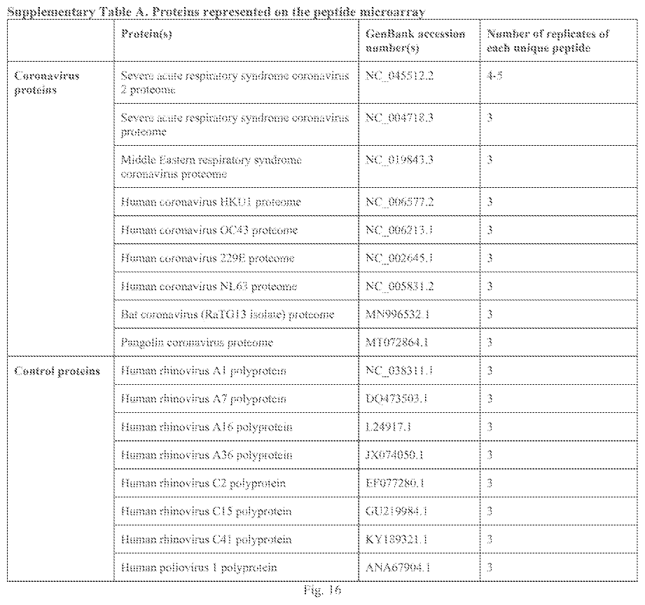

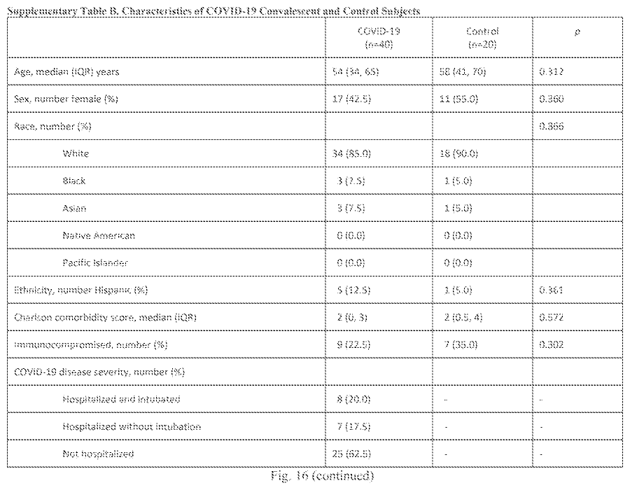

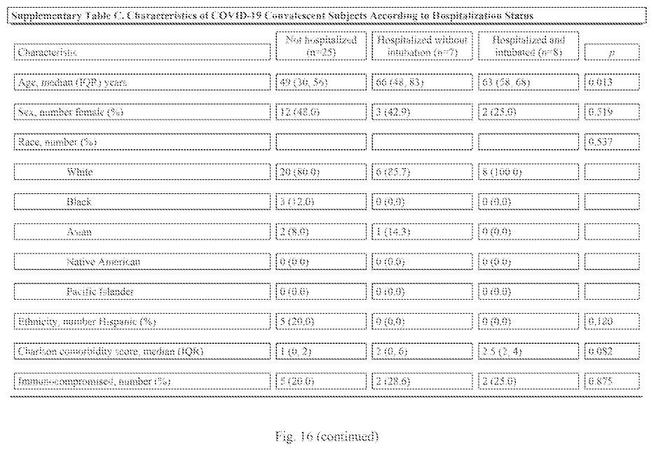

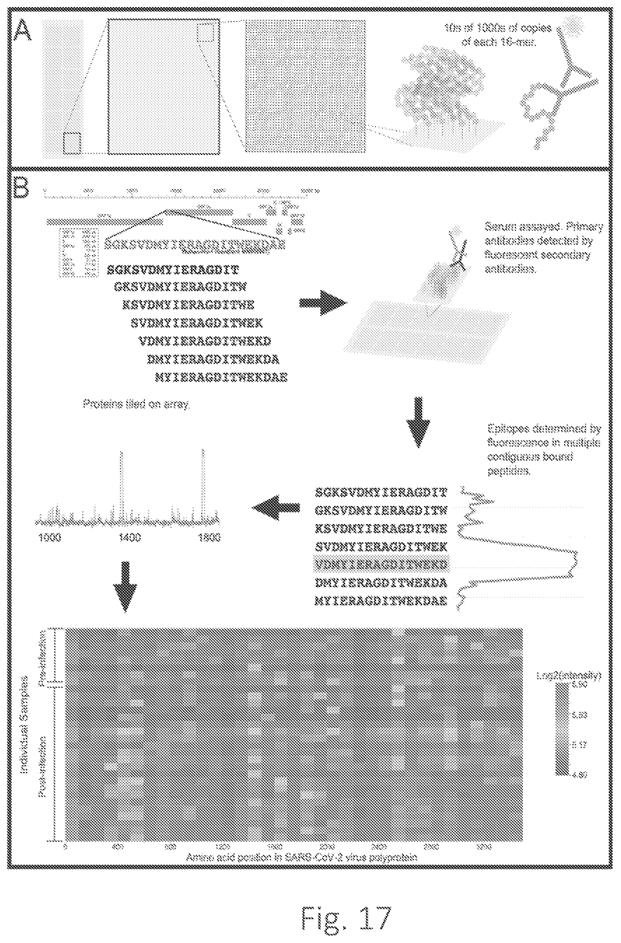

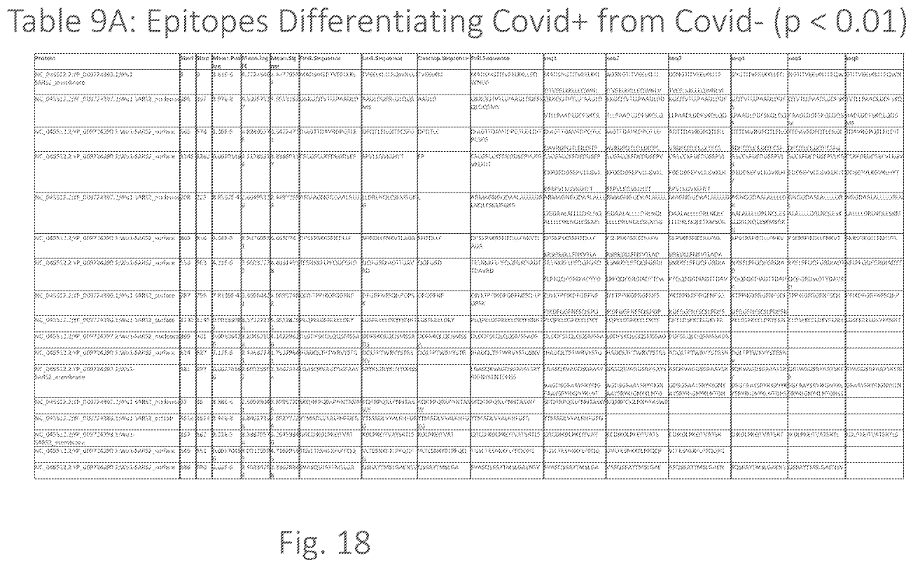

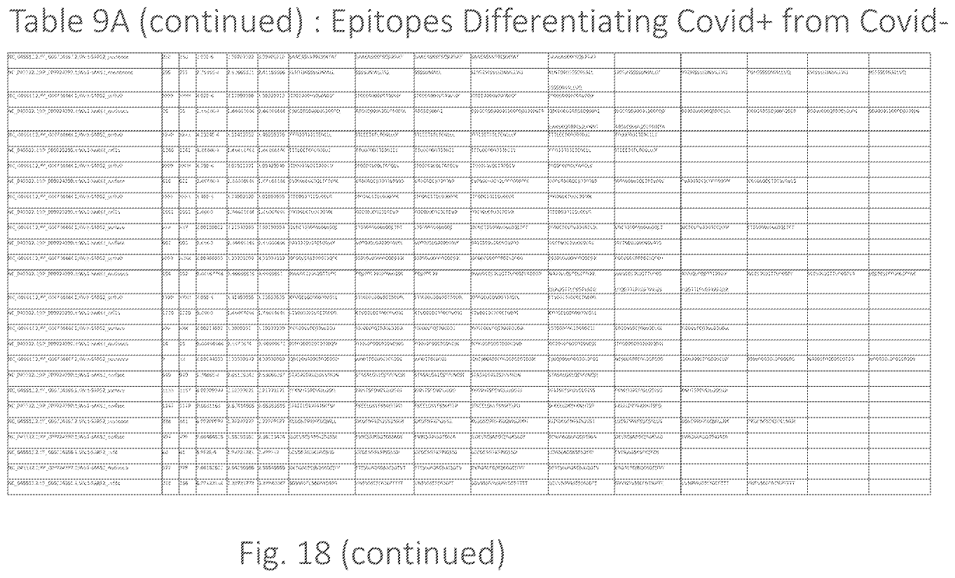

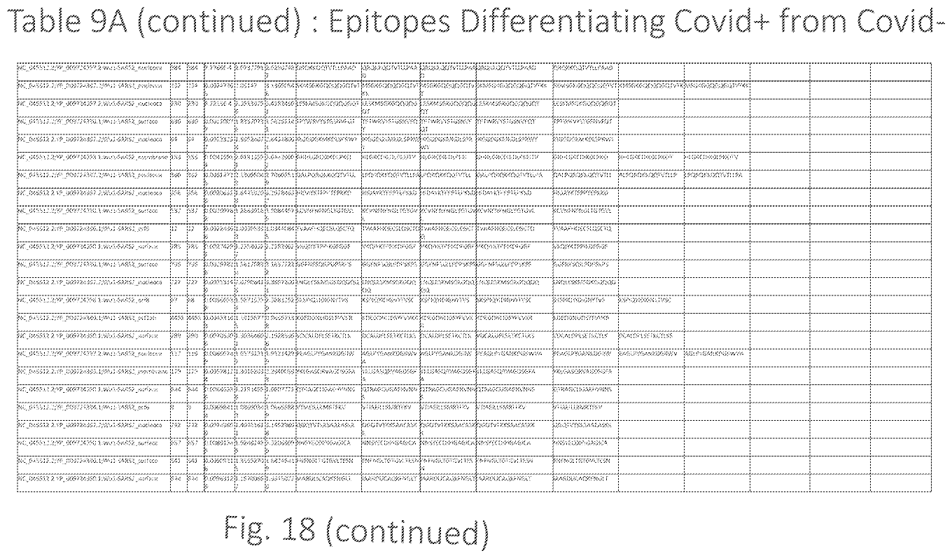

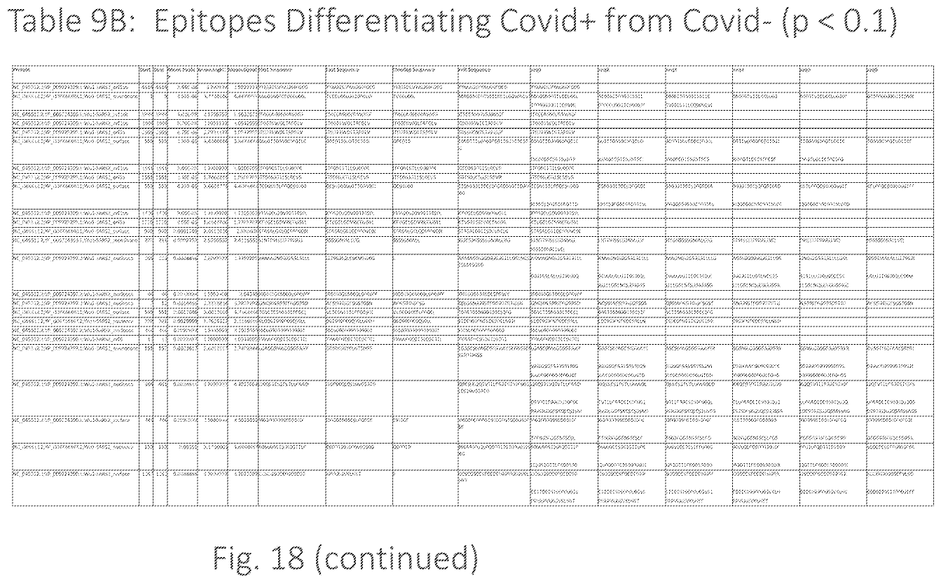

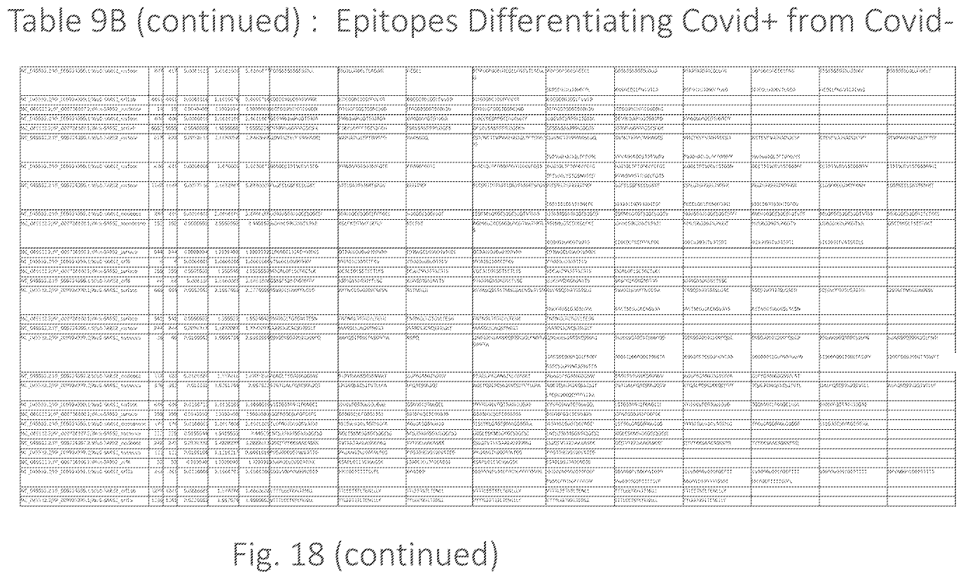

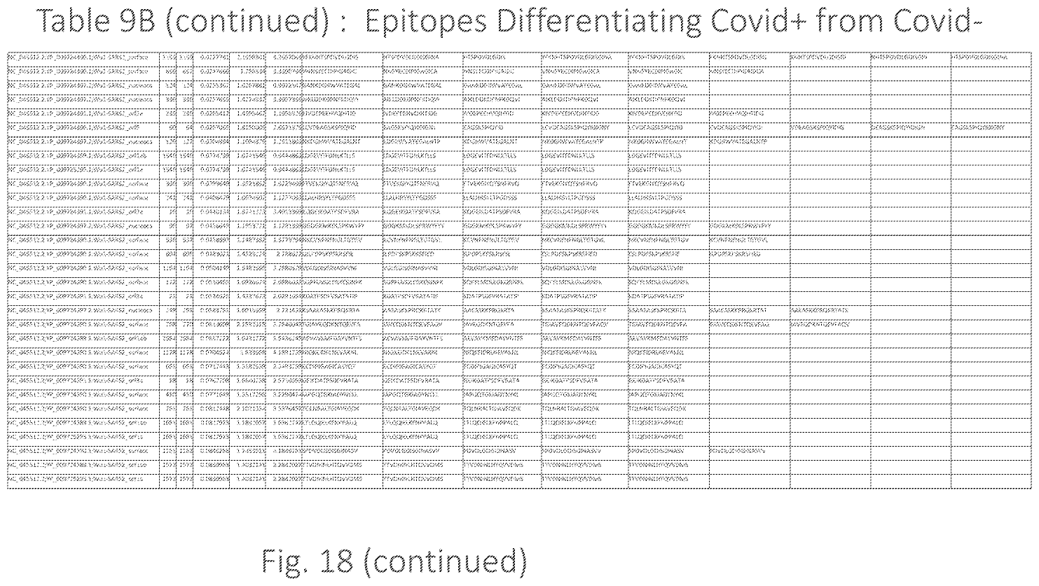

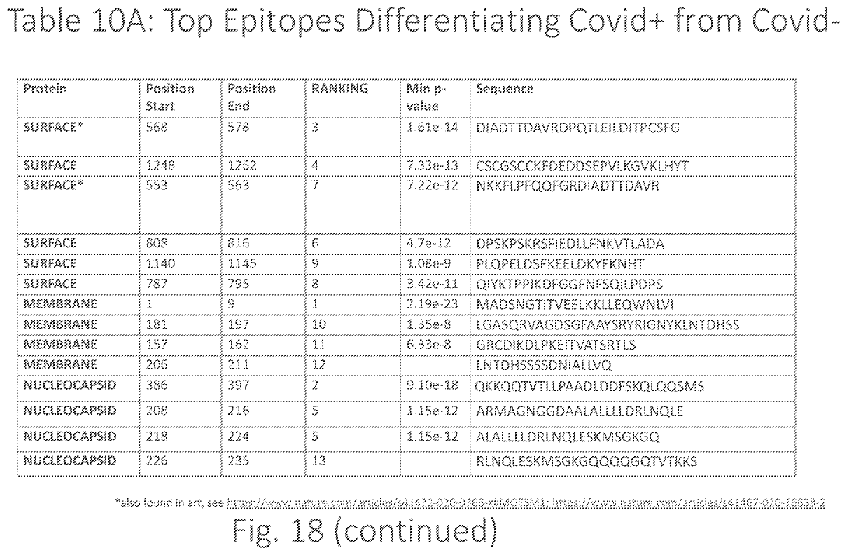

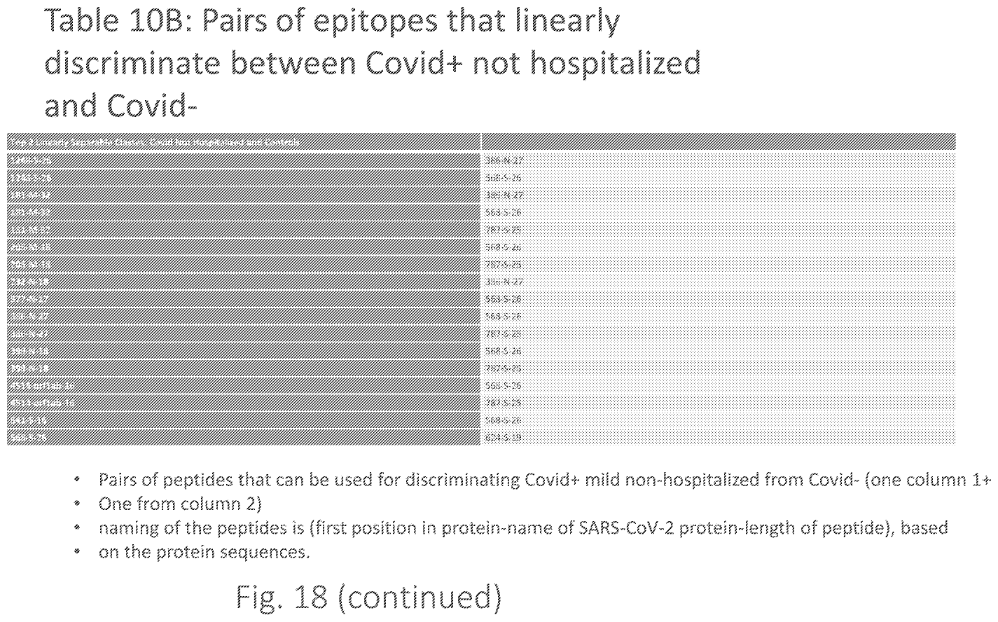

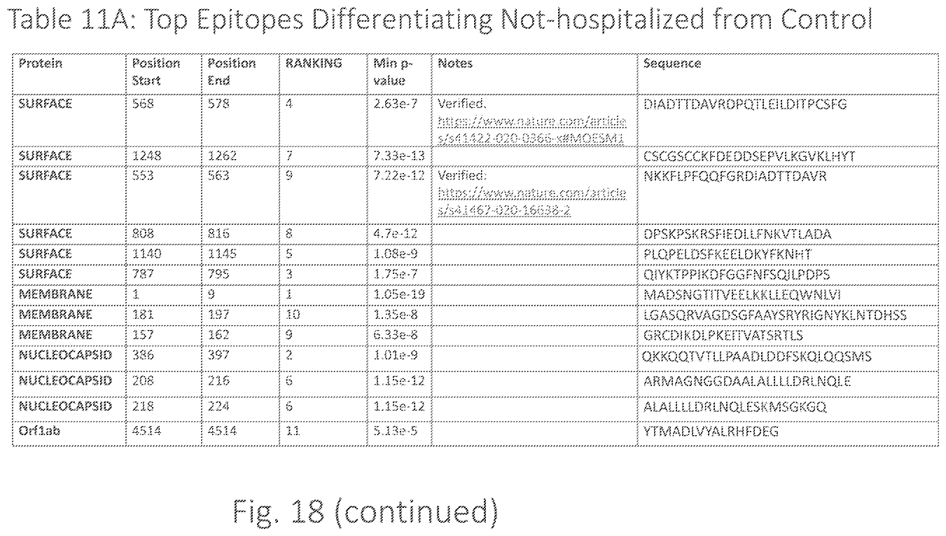

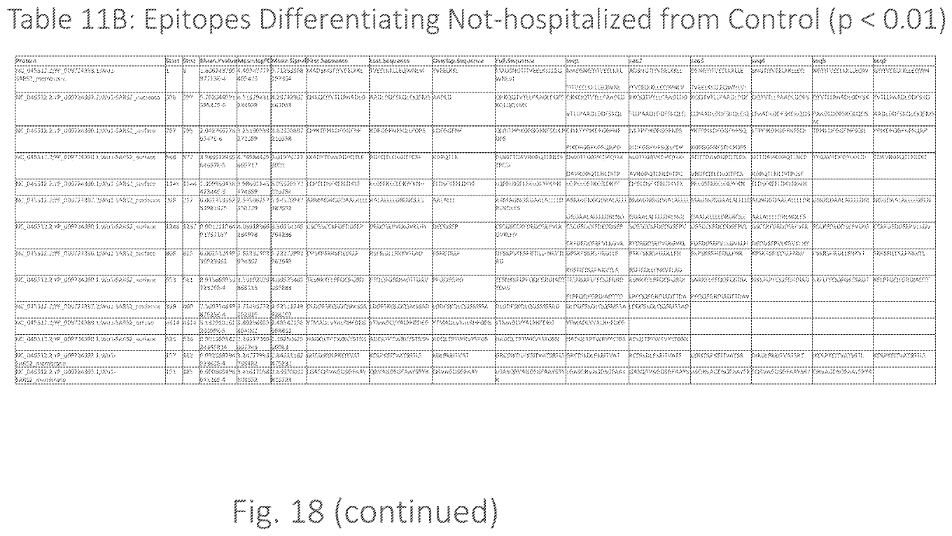

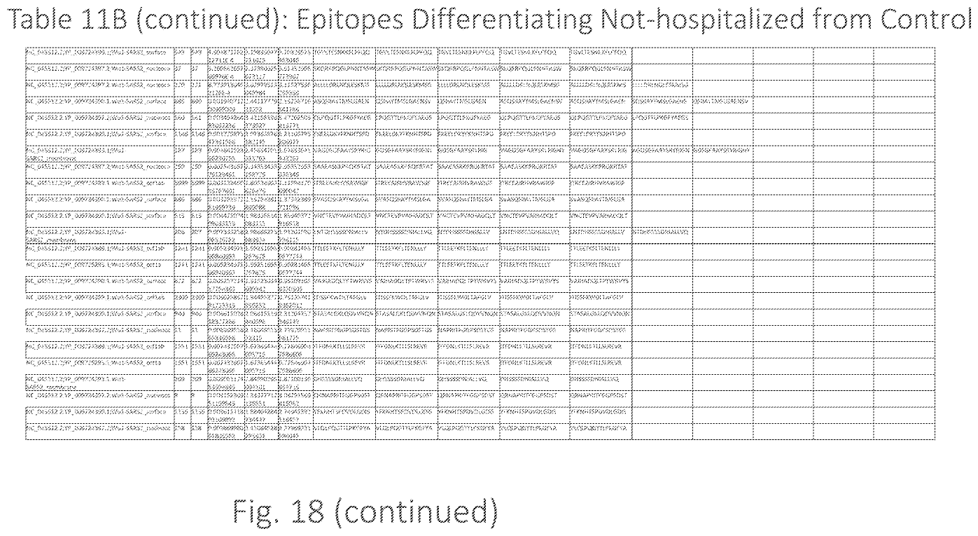

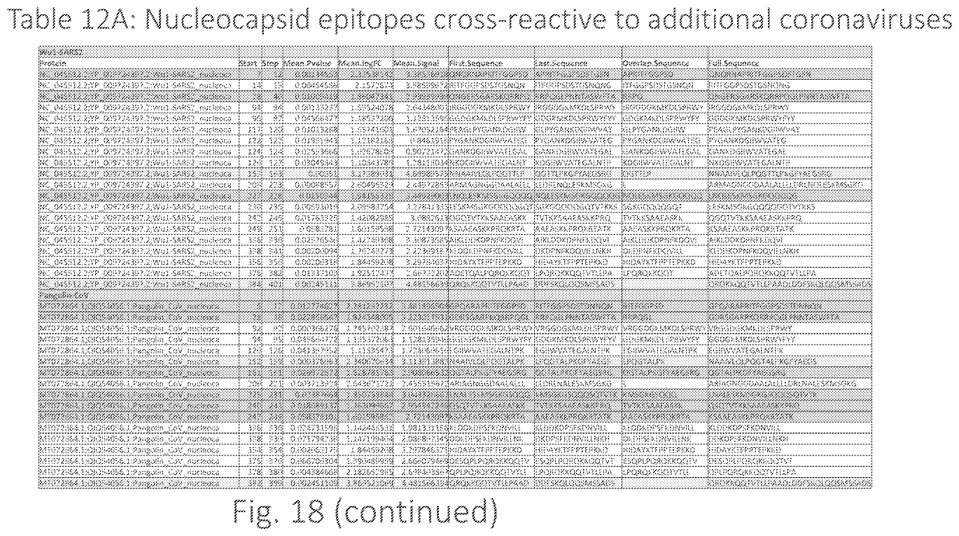

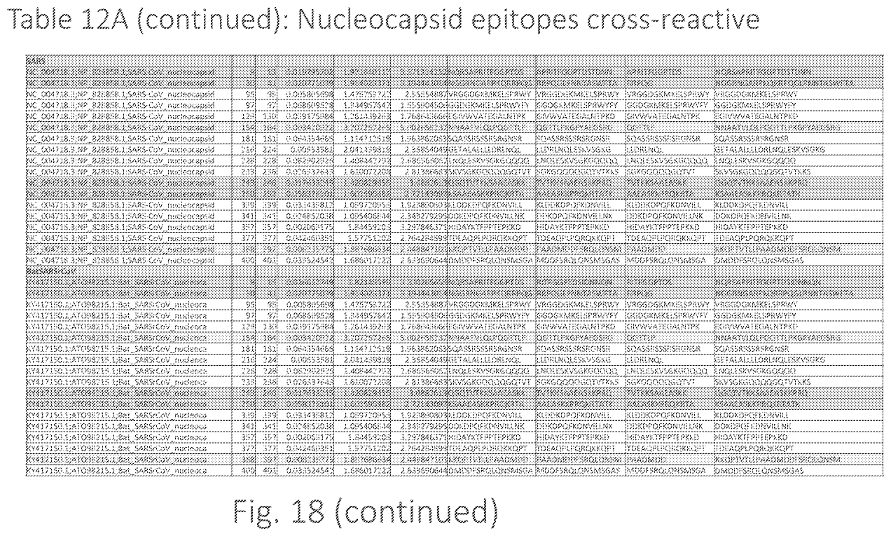

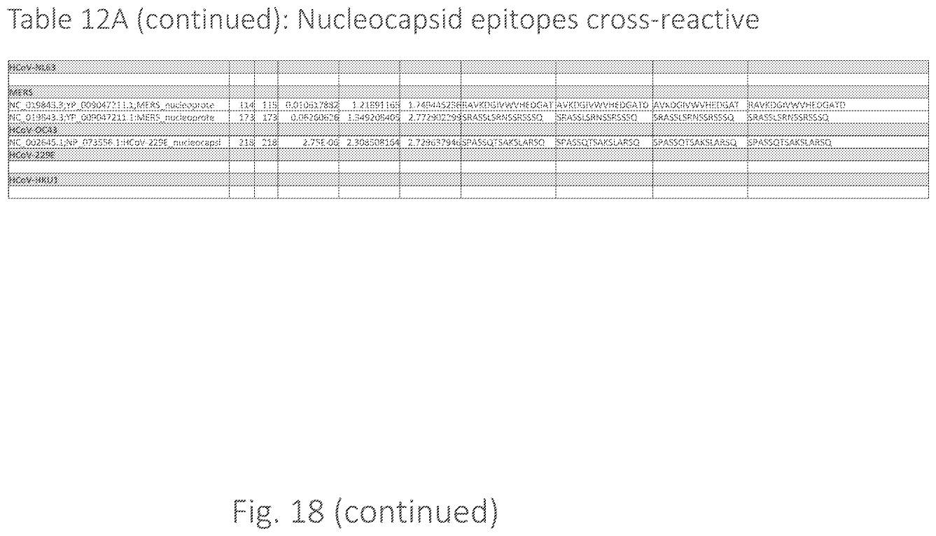

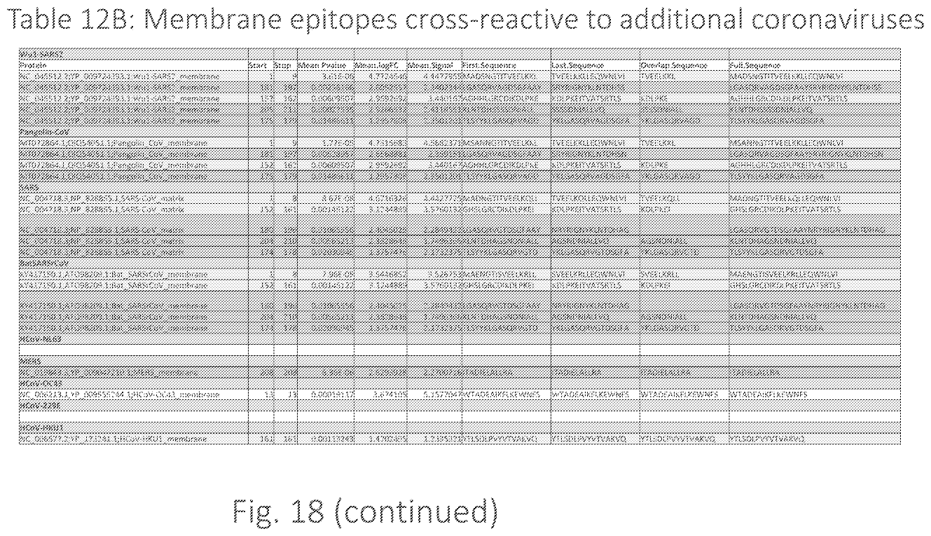

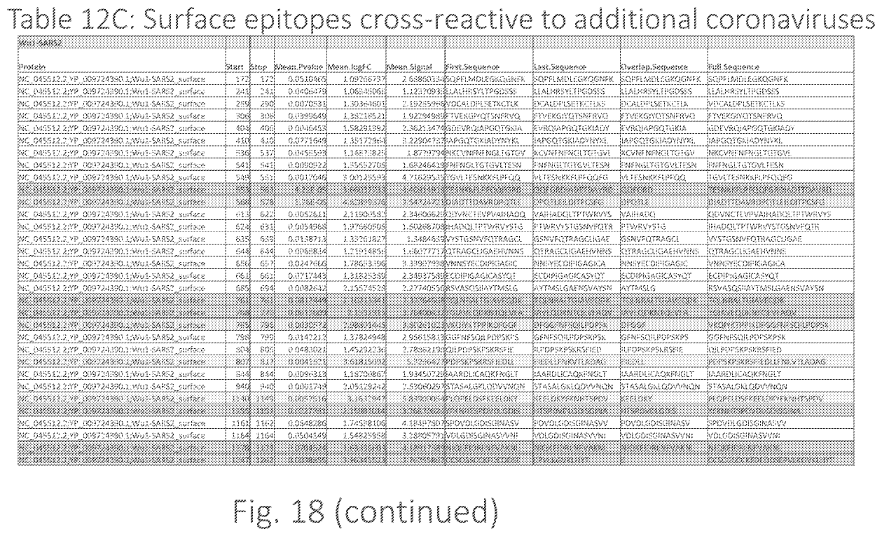

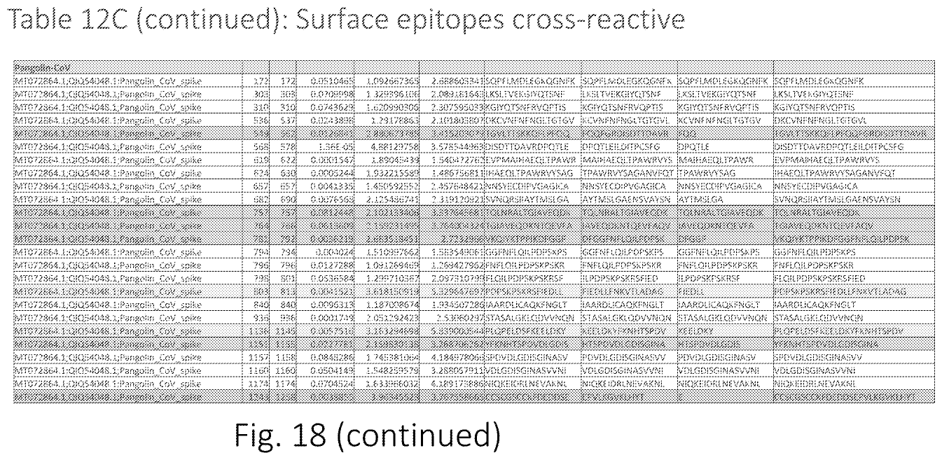

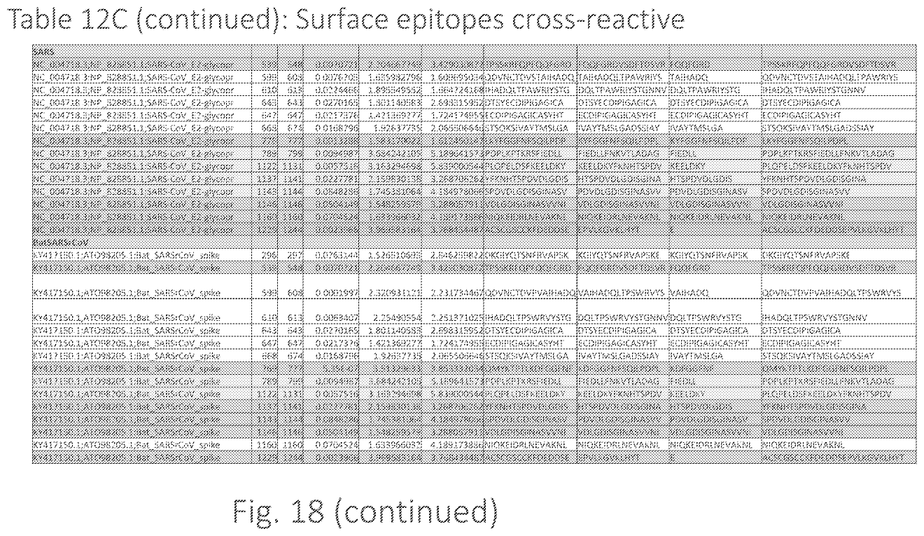

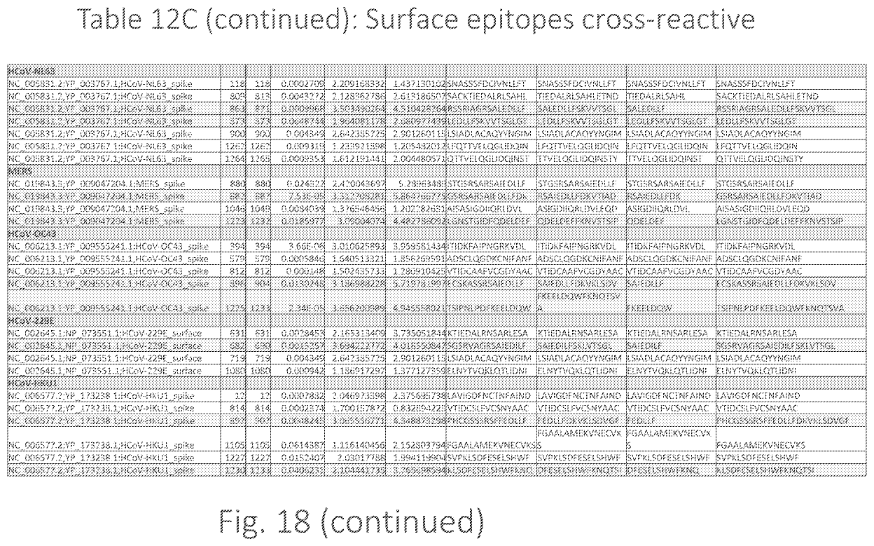

Antibodies in COVID-19-convalescent sera appeared to be cross-reactive with identical or homologous epitopes in S, M. N, ORFlab, ORF3, ORF6, and ORF8 in other CoVs (FIG. 5, FIG. 10, and FIGS. 14 and 15 (Table 6 and 7)). Overall, the greatest number of epitopes in any non-SARS-CoV-2 CoV occurred in the RaTG13 bat β-CoV at 74 epitopes (60 identical to SARS-CoV-2, 13 homologous nonidentical, 1 without a homologous SARS-CoV-2 epitope). The second greatest number, 60 epitopes, occurred in the pangolin CoV (23 identical to SARS-CoV-2, 30 homologous nonidentical, 6 without a homologous SARS-CoV-2 epitope, 1 without a homologous region in SARS-CoV-2), and third SARS-CoV with 45 epitopes, (10 identical to SARS-CoV-2, 32 homologous nonidentical, 3 without a homologous SARS-CoV-2 epitope) (FIGS. 14 and 15, Tables 6 and 7). Most (8 of 12) of the epitopes that were not in areas having epitopes in the homologous SARS-CoV-2 region occurred in ORFlab, with the others occurring in S (2 epitopes) and N (2 epitopes). These epitopes were not conserved among each other and were not conserved with any epitopes in the CCCoVs (FIG. 14. Table 6).

One region, corresponding to SARS-CoV-2 epitope 807-S-26, showed binding or potential cross-reactivity across all CoVs. and one, corresponding to SARS-CoV-2 epitope 1 140-5-25, showed binding or potential cross-reactivity across all β-CoVs (FIG. 5). Epitope 807-5-26 includes the CoV S fusion peptide, and 1140-5-25 is immediately adjacent to the heptad repeat region 2, both of which are involved in membrane fusion.

Enzyme-Linked Immunosorbent Assays (ELISAs) Confirm Peptide Microarray Findings

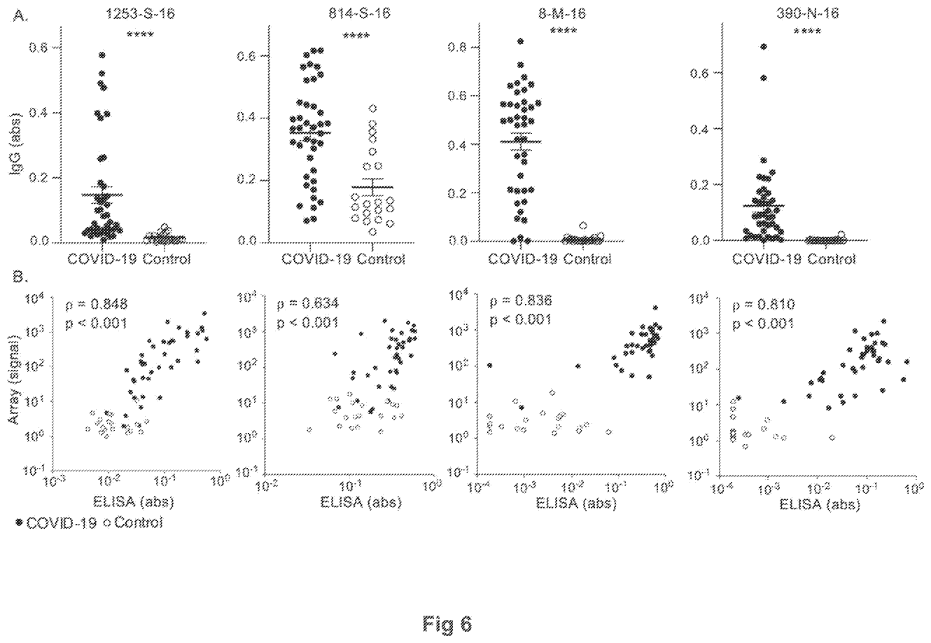

Having determined reactivity and apparent cross-reactivity by peptide array, we aimed to independently confirm and validate these findings by ELISA. We selected 4 peptides for ELISA evaluation (1253-5-16, 814-S-16, 8-M-16, and 3′)0-N-16) from those in our top 10 ranked epitopes, considering diversity among the proteins represented, association with neutralizing capacity, and potential cross-reactivity across multiple CoVs, and using the 16-mer in each epitope that most correctly discriminated between patients and controls. All 4 SARS-CoV-2 peptides had higher IgG binding in COVID-19 convalescent sera than in controls (FIG. 6). Peptide 8-M-16 showed the greatest discrimination between COVID-19 convalescent and control sera with only 3 COVID-19 convalescent samples having values similar to controls. Both peptides 1253-S-16 and 814-S-16 showed greater binding in controls than either 8-M-16 or 3′9)-N-16, confirming our findings of greater potential cross-reactivity among epitopes found in S.

Reactivity in Some Epitopes Correlates with Disease Severity

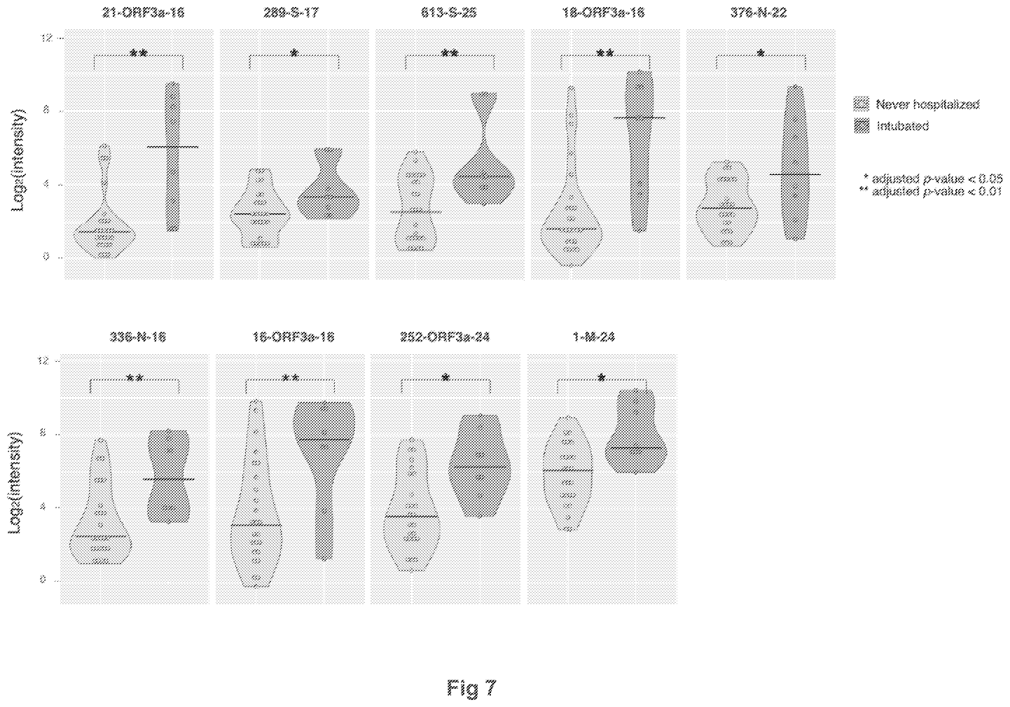

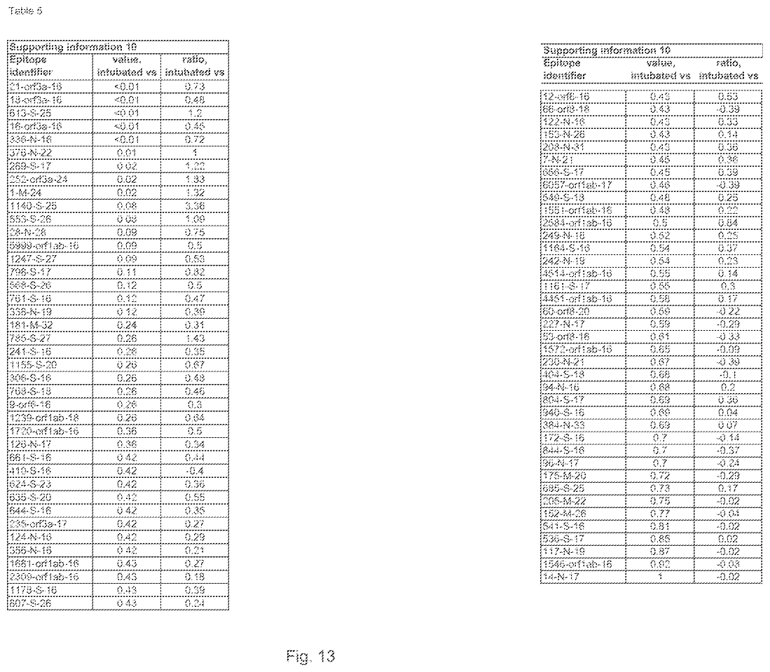

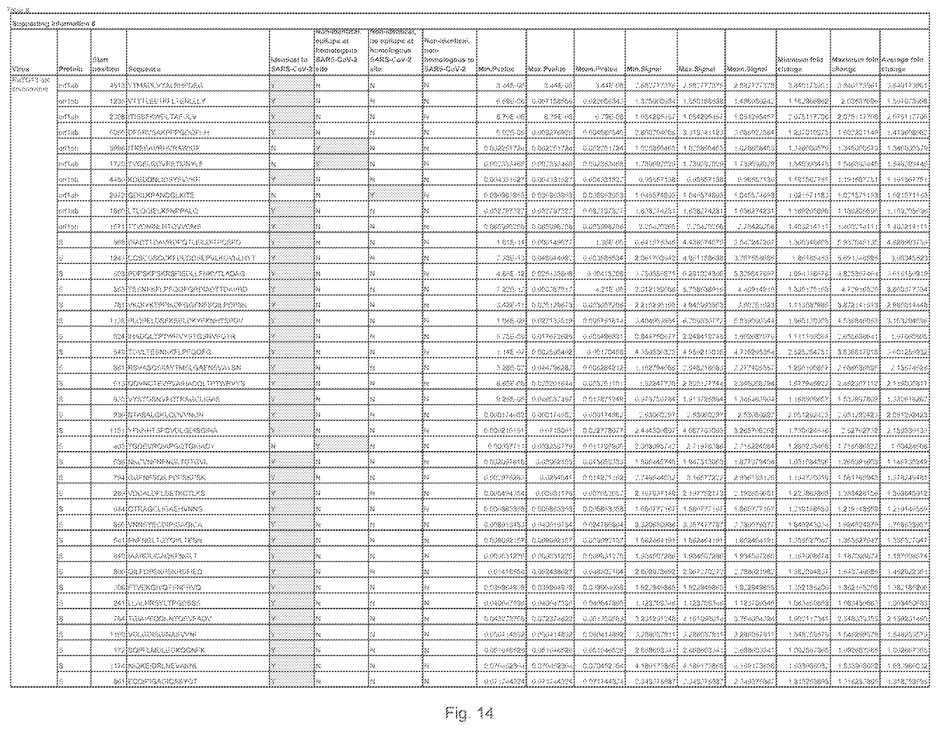

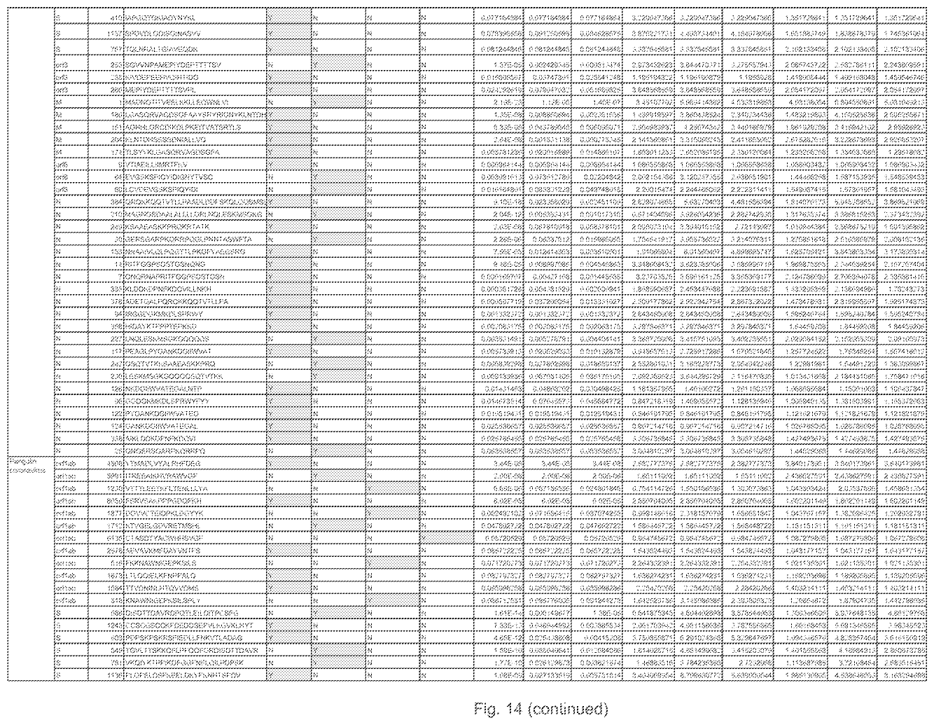

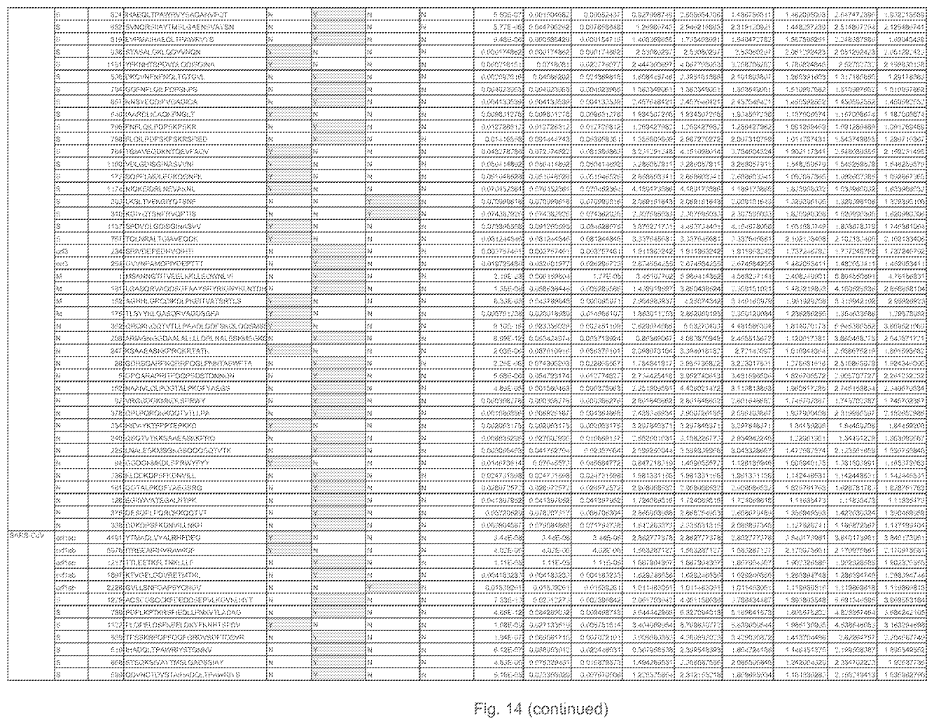

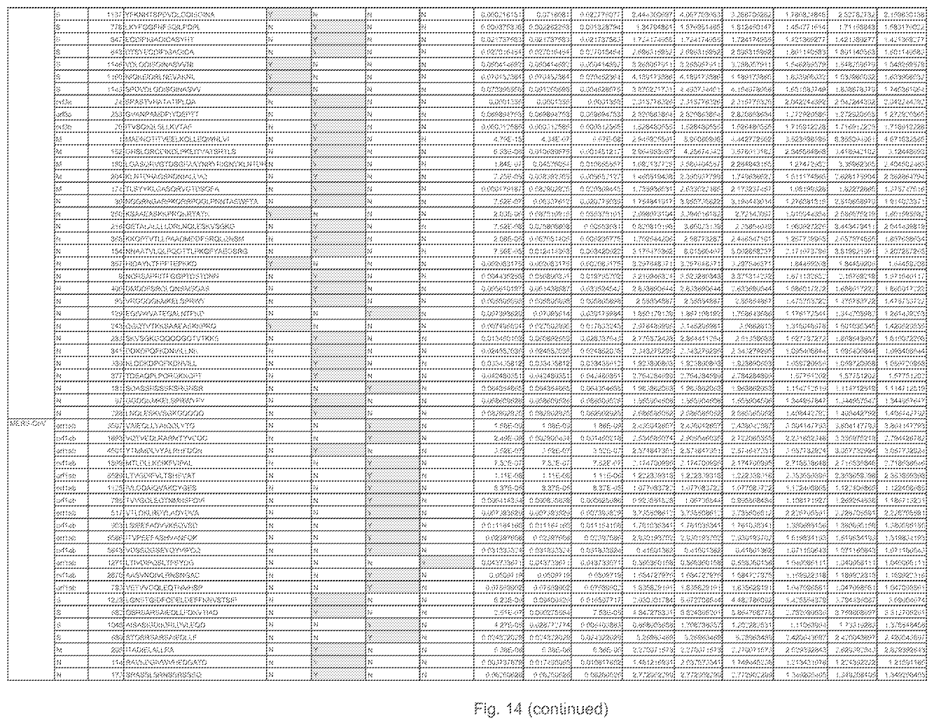

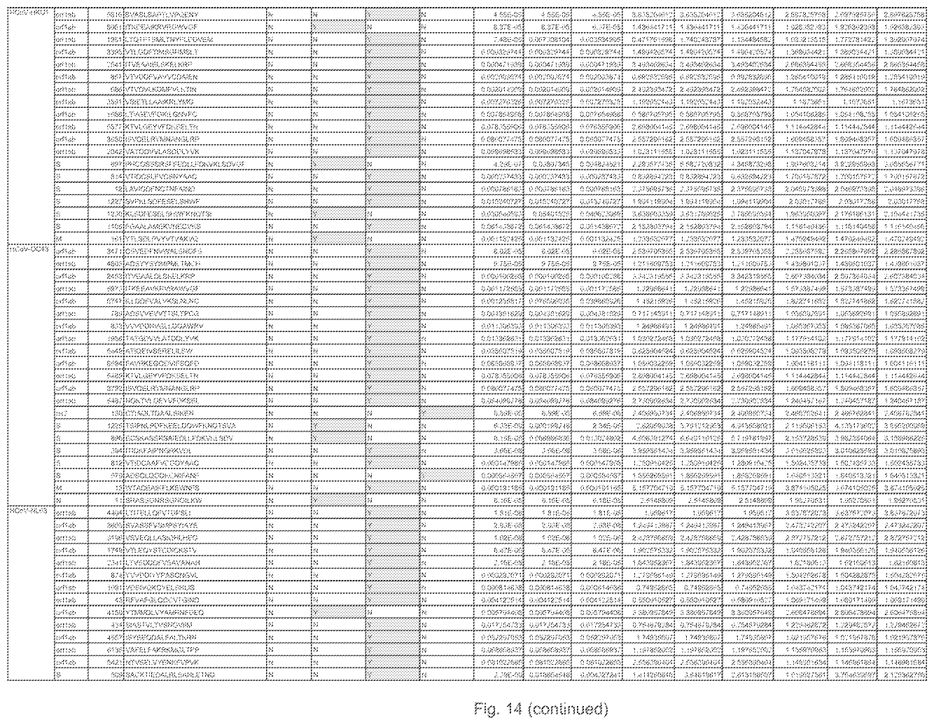

Increased antibody titer and duration have been associated with increased severity of illness due to infection with SARS-CoV-2 and other CoVs, although data on epitope-level differences by severity is lacking. We compared reactivity in patients within our cohort whose COVID-19 course required intubation and mechanical ventilation (n=8) with reactivity in COVID-19 convalescent patients who never required hospitalization (n=25) using multilinear regression accounting for age, sex, immunocompromising conditions, and Charlson comorbidity index score to determine epitope-level resolution of differences in reactivity. Nine epitopes in S (2 epitopes), M (1 epitope), N (2 epitopes), and ORF3a (4 epitopes) showed statistically significant (p<0.05) increases in reactivity for intubated patients relative to never-hospitalized patients (FIG. 7. FIG. 13 (Table 5)). The S epitopes (289-S-17 and 613-S-25) both occurred in the S1 subunit (aa 14-685), with one (289-S-17) in the N-terminal domain (see FIG. 4D), whose function is not well understood but which may play a role in membrane fusion. The M epitope (1-M-24) was the highly reactive epitope in the N-terminus of this protein discussed above. The N epitopes (336-N-16 and 376-N-22) occurred in the C-terminal domain (336-N-16), which is thought to bind nucleic acids, and in the unstructured C-tail (376-N-22). The ORF3a epitopes clustered near the N-terminus of the protein (16-ORF3a-16, 18-ORF3a-16, and 21-ORF3a-16) with one other epitope nearer the C-terminus (252-ORF3a-24). No epitopes showed statistically significant increases in reactivity for never-hospitalized patients relative to intubated patients (FIG. 13 (Table 5)).

### DISCUSSION

In our analysis of antibody binding to the full proteome of SARS-CoV-2, the highest magnitude binding of anti-SARS-CoV-2 antibodies from human sera occurred for an epitope in the N-terminus of M protein, with high specificity and sensitivity. Antibodies produced after infection with SARS-CoV-2 reacted with epitopes throughout the proteomes of other human and nonhuman CoVs, recognizing homologous regions across all CoVs. Taken together, these results confirm that humans mount strong, broad antibody responses to SARS-CoV-2 proteins in addition to S and N, and they implicate M epitopes as highly relevant to diagnostic and potentially to vaccine design.

M proteins are the most abundant proteins in CoV virions. The N-terminus of M is known in other CoVs to be a small, glycosylated ectodomain that protrudes outside the virion and interacts with S, N, and E, while the rest of M resides within the viral particle. Full-length SARS-CoV M has been shown to induce protective antibodies, and patterns of antibodies binding to SARS-CoV M are similar to those we found in SARS-CoV-2. SARS-CoV anti-M antibodies can synergize with anti-S and anti-N antibodies for improved neutralization, and M has been used in protective SARS-CoV and MERS-CoV vaccines. However, the mechanism of protection of anti-M antibodies remains unknown, and this protein remains largely understudied and underutilized as an antigen. Other groups have not previously identified the high magnitude binding we observed for M, though that may be due to other studies' use of samples collected earlier in the course of infection or different techniques, populations, or computational algorithms. Notably, some of the highest binding we observed in the S protein occurred at the base of the extracellular portion of the protein, which would be the site of the putative interaction between SARS-CoV-2 S and M. The ACE2 binding site and the RBD in general are not as reactive, by these methods, as expected, suggesting that other, less-investigated epitopes may be playing a larger role in immunity to SARS-CoV-2 than is currently appreciated, which is further bolstered by the correlation of some of this binding with neutralizing titers. Our results, in concert with prior knowledge of anti-SARS-CoV antibodies, strongly suggest that epitopes in M, particularly the 1-M-24 epitope as well as other novel epitopes we identified, should be investigated further as potential targets in SARS-CoV-2 diagnostics, vaccines, and therapeutics.

Among the accessory proteins against which we detected antibodies, ORF8 has been the best studied. The ORF8 gene is part of a hypervariable region, having undergone multiple substitutions and deletions and being recognized as a recombination hotspot. ORF8 protein is considered to have immunomodulatory activity and has been shown to potently down-regulate major histocompatibility complex class I expression in several cell lines and to antagonize interferon signaling. A deletion in ORF8 appears to be associated with a milder clinical COVID-19 course, and ORF8 has been shown to be secreted, indicating that the epitopes we defined here may merit further investigation for development of potential vaccines and therapeutics. Less is known about the other SARS-CoV-2 accessory proteins in which we found epitopes, ORF3a and ORF6, although some studies have implicated them in immunomodulatory functions.

Interestingly, we found antibodies bind or bind adjacent to a number of the mutations in some of the “variants of concern” (VOCs) of SARS-CoV-2, which have recently emerged, so named because they appear to potentially be more transmissible than previous known variants or to escape antibody binding. The epitopes we defined contained or were immediately adjacent to the locations of the majority of the variant-defining mutation sites in the structural and accessory proteins of the B.1.1.7 and B.1.351 variants and to one-quarter of the structural or accessory protein mutation sites of the P.1, B.1.427, and B.1.429 variants (see FIG. 16 (Table 8)). These results suggest that antigen escape may be driving the rise and dominance of variants. Recent works have demonstrated this phenomenon with mutations in S but have not investigated this possibility for other proteins. Our findings suggest that antibodies to non-S proteins may be important to this process, as well.

We also found that antibodies produced in response to SARS-CoV-2 infection appear to bind peptides representing homologous epitopes throughout the proteomes of other human and nonhuman CoVs. Hundreds of CoVs have been discovered in bats and other species, making future spillovers inevitable. The potential broad cross-reactivity we observed in some homologous peptide sequences may help guide the development of pan-CoV vaccines, especially given that antibodies binding to 807-S-26 and 1140-S-25, which showed potential cross-reactivity across all CoVs and all β-CoVs, respectively, are known to be potently neutralizing. A caveat is that our methods cannot discern whether the increased IgG binding to CCCoVs in COVID-19 convalescent sera is due to newly developed cross-reactive antibodies or due to the stimulation of a memory response against the original CCCoV antigens. However, cross-reactivity of anti-SARS-CoV-2 antibodies with SARS-CoV or MERS-CoV is likely real, since our population was very unlikely to have been exposed to those viruses. Further, our methods efficiently detect antibody binding to linear epitopes, but their sensitivity for detecting parts of conformational epitopes, which are considered highly important in the immune response to SARS-CoV-2 and which are believed to be the type of epitope found within the RBD, is unknown. It is interesting to note that SARS-CoV-2 infection resulted in some antibodies that bound epitopes in other coronaviruses without binding the homologous part of SARS-CoV-2. These epitopes were not conserved with each other (FIG. 14), and most of this binding occurred in nonstructural proteins in ORFlab, which may indicate that this was nonspecific binding resulting from a generalized immune activation.

Finally, we demonstrated that more severely ill patients have significantly greater reactivity to certain epitopes in S, M, N, and ORF3a. The 9 epitopes with significantly higher magnitude reactivity in intubated patients may play a role in the overaggressive immune response known to characterize severe COVID-19, suggesting that they may be targets for treatment in or prevention of severe disease. Our data collection included date of first positive test but not of symptom onset, but future studies that include these data could investigate potential correlations between symptoms and antibody kinetics. Alternatively, the antibody response in general may be higher in very sick patients. expanding the repertoire of antibody reactivity. Future studies should investigate whether these differences can be detected early in the disease course to determine their potential utility as predictive markers of disease severity. Future studies may also investigate these epitopes' potential as targets for medical countermeasures, although consideration should be given to the small sample size of our investigation.

Our extensive profiling of epitope-level resolution antibody reactivity in COVID-19 convalescent patients, confirmed by independent assays, provides new epitopes that could serve as important targets in the development of improved diagnostics, vaccines, and therapeutics against SARS-CoV-2 and dangerous human CoVs that may emerge in the future.

Methods

Peptide Microarray Design and Synthesis

Viral protein sequences were selected and submitted to Nimble Therapeutics (Madison, Wisconsin, USA) for development into a peptide microarray (Heffron A S, Mohr E L, Baker D, Haj A K, Buechler C R, Bailey A. et al. Antibody responses to Zika virus proteins in pregnant and non-pregnant macaques. PLoS Negl Trop Dis. 2018; 12: e0006903. pmid: 30481182). Sequences represented include proteomes of all 7 coronaviruses known to infect humans, proteomes of closely related coronaviruses found in bats and pangolins, and spike proteins from other coronaviruses (accession numbers and replicates per peptide shown in FIG. 16 (Table 8)). A number of proteins were included as controls, including poliovirus, 7 strains of human rhinovirus, and human cytomegalovirus 65 kDa phosphoprotein. We chose these controls given that we expect most human adults will have antibody reactivity to at least one of these proteins and proteomes. Accession numbers used to represent each viral protein are listed in the Supporting information (accession numbers and replicates per peptide shown in FIG. 16 (Table 8)). All proteins were tiled as 16 amino acid peptides overlapping by 15 amino acids. All unique peptides were tiled in a lawn of thousands of copies, with each unique peptide represented in at least 3 and up to 5 replicates (FIG. 16). The peptide sequences were synthesized in situ with a Nimble Therapeutics Maskless Array Synthesizer (MAS) by light-directed solid-phase peptide synthesis using an amino-functionalized support (Geiner Bio-One) coupled with a 6-aminohexanoic acid linker and amino acid derivatives carrying a photosensitive 2-(2-nitrophenyl) propyloxycarbonyl (NPPOC) protection group (Orgentis Chemicals). Unique peptides were synthesized in random positions on the array to minimize impact of positional bias. Each array consists of 12 subarrays, where each subarray can process 1 sample, and each subarray contains up to 389,000 unique peptide sequences.

Human Patients and Controls

The study was conducted in accordance with the Declaration of Helsinki and approved by the Institutional Review Board of the University of Wisconsin-Madison. Clinical data and sera from patients infected with SARS-CoV-2 were obtained from the University of Wisconsin (UW) COVID-19 Convalescent Biobank and from controls (sera collected prior to 2019) from the UW Rheumatology Biobank. All patients and controls were 18 years of age or older at the time of recruitment and provided informed consent. COVID-19 convalescent patients had a positive SARS-COV-2 PCR test at UW Health with sera collected 5 to 6 weeks after self-reported COVID-19 symptom resolution except blood was collected for 1 patient after 9 weeks. Age, sex, medications, and medical problems were abstracted from UW Health's electronic medical record (EMR). Race and ethnicity were self-reported. Hospitalization and intubation for COVID-19 and smoking status at the time of blood collection (controls) or COVID-19 were obtained by EMR abstraction and self-report and were in complete agreement. Two-thirds of COVID-19 convalescent patients and all controls had a primary care appointment at UW Health within 2 years of the blood draw as an indicator of the completeness of the medical information. Patients and controls were considered to have an immunocompromising condition if they met any of the following criteria: immunosuppressing medications, systemic inflammatory or autoimmune disease, cancer not in remission, uncontrolled diabetes (secondary manifestations or hemoglobin A1c>7.0%), or congenital or acquired immunodeficiency. Controls and COVID-19 patients were similar in regard to demographics and health (FIG. 16), and patients who were not hospitalized, were hospitalized, or were hospitalized and intubated also were compared (FIG. 16). No patients or controls were current smokers.

Peptide Array Sample Binding

Samples were diluted 1:100 in binding buffer (0.01 M Tris-Cl (pH 7.4), 1% alkali-soluble casein, 0.05% Tween-20) and bound to arrays overnight at 4° C. After sample binding, the arrays were washed 3× in wash buffer (1× TBS, 0.05% Tween-20), 10 minutes per wash. Primary sample binding was detected via Alexa Fluor 647-conjugated goat anti-human IgG secondary antibody (Jackson ImmunoResearch, West Grove, Pennsylvania, United States of America). The secondary antibody was diluted 1:10,000 (final concentration 0.1 ng/μl) in secondary binding buffer (1× TBS, 1% alkali-soluble casein, 0.05% Tween-20). Arrays were incubated with secondary antibody for 3 hours at room temperature, then washed 3× in wash buffer (10 minutes per wash), washed for 30 seconds in reagent-grade water, and then dried by spinning in a microcentrifuge equipped with an array holder. The fluorescent signal of the secondary antibody was detected by scanning at 635 nm at 2 μm resolution using an Innopsys 910AL microarray scanner. Scanned array images were analyzed with proprietary Nimble Therapeutics software to extract fluorescence intensity values for each peptide.

Peptide Microarray Findings Validation

We included sequences on the array of viruses that we expected all adult humans to be likely to have been exposed to as positive controls: 1 poliovirus strain (measuring vaccine exposure) and 7 rhinovirus strains. Any patient or control whose sera did not react to at least 1 positive control would be considered a failed run and removed from the analysis. All patients and controls in this analysis reacted to epitopes in at least 1 control strain (FIG. 1).

Peptide Microarray Data Analysis

The raw fluorescence signal intensity values were log₂ transformed. Clusters of fluorescence intensity of statistically unlikely magnitude, indicating array defects, were identified and removed. Local and large area spatial corrections were applied, and the median transformed intensity of the peptide replicates was determined. The resulting median data was cross-normalized using quantile normalization.

Neutralization Assay

Virus neutralization assays were performed with SARS-CoV-2/UW-001/Human/2020/Wisconsin on Vero E6/TMPRSS2. Virus (approximately 100 plaque-forming units) was incubated with the same volume of 2-fold dilutions of heat-inactivated serum for 30 minutes at 37° C. The antibody/virus mixture was added to confluent Vero E6/TMPRSS2 cells that were plated at 30,000 cells per well the day prior in 96-well plates. The cells were incubated for 3 days at 37° C. and then fixed and stained with 20% methanol and crystal violet solution. Virus neutralization titers were determined as the reciprocal of the highest serum dilution that completely prevented cytopathic effects.

Protein Structures

The SARS-CoV-2 S-chimera.pdb used to make S protein structures is a chimeric structure built by Robert Kirchdoerfer using 6VYB.pdb, 5X4S.pdb, and 6LZG coordinates and filling in internal unresolved residues from known (presumably) analogous sites determined for SARS-CoV S from 6CRV.pdb. Additional unmodeled regions were generated using Modeller. C-proximal HR2 regions were modeled as single helices (Phe1148-Leu1211) in Coot.

The data2bfactor Python script written by Robert L. Campbell. Thomas Holder, and Suguru Asai (downloaded from pldserver1.biochem.queensu.ca/˜rlc/work/pymol/) used to substitute peptide array data onto this structure in place of the B factor in PyMol (The PyMOL Molecular Graphics System. Version 2.0 Schrödinger, LLC) using a dark blue (low) to red (high) color scale. Data used for these visualizations were the average reactivity in the 40 COVID-19 convalescent patients, the average reactivity in the 20 naïve controls, and the difference between averages for the patients and for the controls.

Enzyme-Linked Immunosorbent Assays (ELISAs)

Costar 96-well high-binding plates (Corning. Coming, USA) were incubated at 4° C. overnight with 5 μg/ml streptavidin (Thermo Fisher Scientific, Waltham, USA) in PBS (Corning). Plates were washed twice with PBS and incubated at room temperature for 1 hour with 0.5 mM of the following peptides (Biomatik. Kitchener, Canada) in PBS: 814-S-16 (KRSFIEDLLFNKVTLA-K-biotin (SEQ ID NO:96), 1253-S-16 (CCKFDEDDSEPVLKGV-K-biotin (SEQ ID NO:10)), 390-N-16 (QTVTLLPAADLDDFSK-K-biotin (SEQ ID NO:3)), and 8-M-16 (TTVEELKKLLEQWNLV-K-biotin (SEQ ID NO:1)). Plates were washed thrice with wash buffer (0.2% Tween-20 in PBS), then incubated for 1 hour in blocking solution (5% nonfat dry milk in wash buffer) at room temperature, incubated overnight at 4° C. with sera at 1:200 in blocking solution, washed 4 times with wash buffer, incubated for 1 hour at room temperature with mouse anti-human IgG conjugated to horse radish peroxidase (Southern Biotech, Birmingham, USA) diluted 1:5,000 in blocking solution, washed 4 times with wash buffer, and incubated with tetramethyl benzidine substrate solution (Thermo Fisher Scientific) for 5 minutes followed by 0.18 M sulfuric acid. Absorbance was read on a FilterMax F3 Multi-mode Microplate reader (Molecular Devices, San Jose, USA) at 450 and 562 nm. Background signal from 562 nm absorbance and wells with no peptide and no serum were subtracted. Plates were normalized using a pooled serum sample on every plate. Absorbance values of 0 were plotted as 0.0002 to allow a log scale for graphs. Samples were run in duplicate.

Statistical Analysis

Statistical analyses were performed in R (v 4.0.2) using in-house scripts. For each peptide, a p-value from a two-sided t test with unequal variance between sets of patient and control responses were calculated and adjusted using the Benjamini-Hochberg (BH) algorithm. To determine whether the peptide was in an epitope (in SARS-CoV-2 proteins) or cross-reactive for anti-SARS-CoV-2 antibodies (in non-SARS-CoV-2 proteins), we used an adjusted p-value cutoff of <0.1 (based on multiple hypothesis testing correction for all 119.487 unique sequences on the array) and a fold-change of greater than or equal to 2 and grouped consecutive peptides as a represented epitope. Linear discriminant analysis leave-one-out cross validation was used to determine specificity and sensitivity on each peptide and from each epitope using the average signal of the component peptides. Pearson correlation for reactivity with neutralizing titer was calculated using each patient's or control's epitope signal and the log 2 signal of the respective neutralization value.

To identify cross-reactive epitopes, we used each SARS-CoV-2 epitope sequence as a query, searched the database of proteins from the sequences in the peptide array using blastp (-word-size 2, num-targets 4.000) to find homologous sequences in the bat, pangolin, and other human CoV strains, then determined whether the average log₂-normalized intensity for these sequences in patients was at least 2-fold greater than in controls with t test statistics yielding adjusted p-values <0.1. Each blast hit was then mapped back to the corresponding probe ranges.

For correlations of reactivity with clinical severity, for each patient, the epitope signal was determined by averaging the normalized signal from the epitopes corresponding probes. Each epitope average signal response was fit using a multilinear regression model accounting for age, sex (Female, Male), immunocompromised status (Yes. No), and Charlson comorbidity index score (Charlson M E, Pompei P. Ales K L, CR MK. A new method of classifying prognostic comorbidity in longitudinal studies: development and validation. J Chronic Dis. 1987; 40:373-83. pmid: 3558716) as additive. Contrasts between nonhospitalized and intubated patients were performed for each epitope with the fit models and p-values and log₂ fold-change were determined.

The clinical and demographic characteristics of convalescent patients were compared to those of the controls using λ2 tests for categorical variables and Wilcoxon rank-sum tests for nonnormally distributed continuous measures Heatmaps were created using the gridtext (Wilke CO. gridtext: Improved Text Rendering Support for ‘Grid’ Graphics. R package version 0.1.1.2020) and complexheatmap (Gu Z, Eils R, Schlesner M. Complex heatmaps reveal patterns and correlations in multidimensional genomic data. Bioinformatics. 2016; 32:2847-9. pmid: 27207943) packages in R. Alignments for heatmaps were created using MUSCLE (Edgar. MUSCLE: multiple sequence alignment with high accuracy and high throughput. Nucleic Acids Res. 2004; 32:1792-7. pmid: 15034147).

### Example 2: Method of Distinguishing Vaccination and Past SARS-CoV-2 Infection

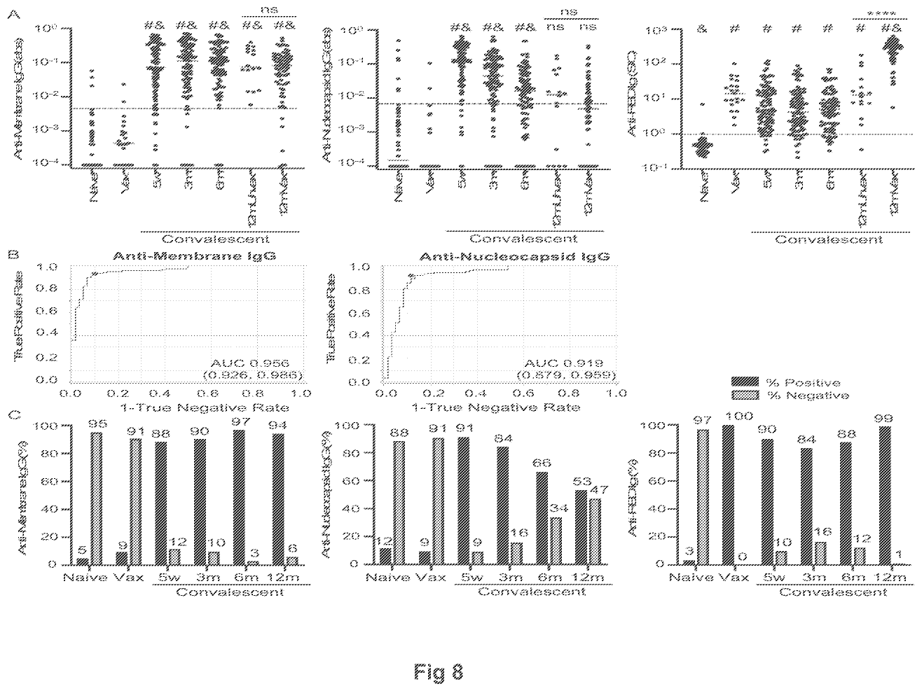

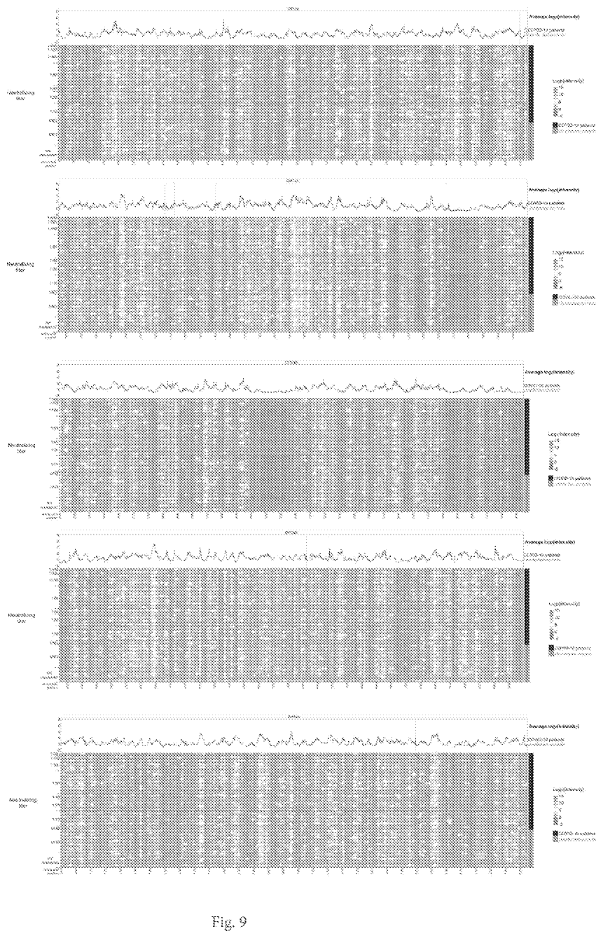

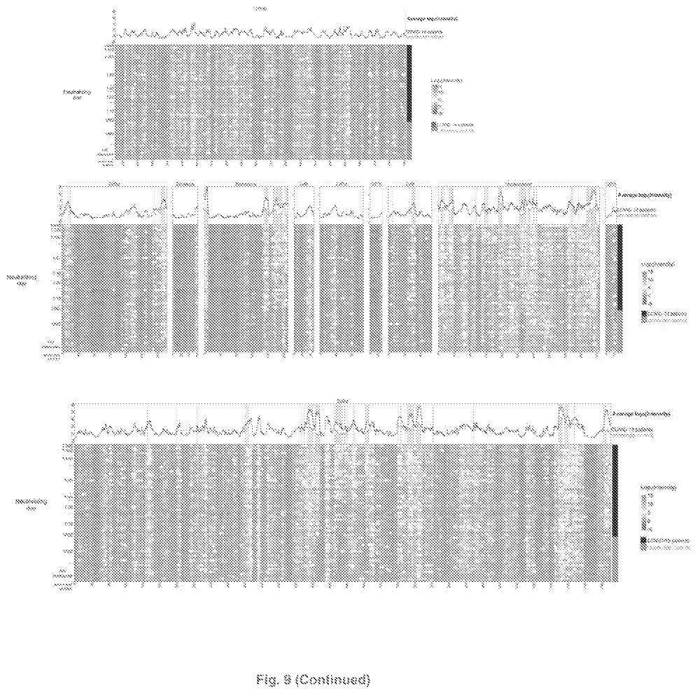

This Example demonstrates that detection of antibodies that bind to the membrane peptides or proteins described herein can distinguish between a subject having been vaccinated to COVID-19 and a subject having past SARS-CoV-2 infection 12 months post-symptom resolution. Currently, the standards for testing for prior infection vs vaccination has been to detect for anti-nucleocapsid antibodies in addition to anti-spike antibodies in sera of an individual. However, as FIG. 8 demonstrates, anti-nucleocapsid peptide antibodies are decreased substantially over time after infection while anti-membrane peptides antibodies remain high and detectable, and suitably correlated with past SARS-CoV-2 infection. Thus, anti-membrane, but not anti-nucleocapsid or anti-spike receptor binding domain antibodies distinguish COVID-19 vaccinated subjects and subjects with past SARS-CoV-2 infection 12 months post-symptom resolution. FIG. 8A shows an ELISA quantification of IgG that binds a biotinylated peptide from SARS-CoV-2 membrane (ITVEELKKLLEQWNLV (SEQ ID NO:1)-K-biotin) and nucleocapsid protein (QTVTLLPAADLDDFSK (SEQ ID NO:3)-K-biotin) with results reported as absorbance (abs). Ig that binds to SARS-CoV-2 spike receptor binding domain (RBD) was detected using Lumit™ Dx SARS-CoV-2 Immunoassay (Promega) with results reported as sample/calibrator (S/C). By 12 months, 73 convalescent subjects had received at least one COVID-19 vaccine dose (12m Vax) and 16 were unvaccinated (12m Unvax). FIG. 8B demonstrates the receiver operating characteristic (ROC) curves generated using ELISA absorbance values for anti-membrane or anti-nucleocapsid IgG with sera from naive and 5w COVID-19 convalescent patients. Area under the curve (AUC) and Wald 95% confidence intervals are in parentheses. FIG. 8C was generated using the cutoffs in (A), the percent positive (black bars) and percent negative (gray bars) results were calculated for naive sera (n=60), sera from vaccinated individuals with no COVID-19 infection (n=21), and 5w (n=121), 3m (n=115), 6m (n=98), and 12m (n=100) COVID-19 convalescent sera.

1. A method of detecting the presence of SARS-CoV-2 antibodies in a sample, the method comprising:
contacting the sample with one or more peptide selected from SEQ ID NO:1-16 and Tables 1, 2, 6, 9-12 or a peptide having at least 90% sequence similarity to a peptide of SEQ ID NO:1-16 or in Tables 1, 2, 6, 9-12; and
detecting the binding of one or more antibodies from the sample to the one or more peptide by a detection agent, wherein the presence of the one or more antibodies within the sample indicates the presence of antibodies against SARS-CoV-2.

2. The method of claim 1, wherein the one or more peptide are selected from SEQ ID NO:1-16 or a peptide with at least 90% sequence similarity to a peptide in SEQ ID NO:1-16.

3. The method of claim 1, wherein one or more peptides are selected from Tables 11A-11B or a peptide with at least 90% sequence similarity to a peptide in Tables 11A-11B.

4. (canceled)

5. The method of claim 1, wherein the contacting step comprises two or more peptides.

6. The method of claim 1, wherein the contacting step comprises at least one membrane protein peptide selected from Table 2 or Tables 11A-11B or a peptide having at least 90% sequence similarity to the peptide in Table 2 or Tables 11A-11B.

7. The method of claim 1, wherein the detection agent is a secondary antibody or fragment thereof, preferably an anti-human IgG antibody or fragment thereof.

8. The method of claim 7, wherein the secondary antibody comprises a detectable tag, preferably wherein the detectable tag is selected from the group consisting of an enzymatic tag, a biotin tag, a chemiluminescent tag and a fluorescent tag.

9. The method of claim 1, wherein the sample is convalescent plasma for the detection of antibodies against SARS-CoV-2.

10. The method of claim 1, wherein the one or more peptide are selected from Tables 12A-12C or a peptide with at least 90% sequence similarity to a peptide in Tables 12A-12C.

11. An immunoassay for detecting antibodies to SARS-CoV-2 in a biological sample, the assay comprising:
a capture agent comprising one or more peptides selected from SEQ ID NO:1-16 and Tables 1, 2, 6 and 9-12 or a peptide with at least 90% similarity to one or more peptides in Tables 1. 2, 6 and 9-12, and
a detection agent capable of binding to the one or more antibodies that bind to the capture agent.

12. The immunoassay of claim 11, wherein the one or more peptides are attached to a solid or semi-solid support.

13. The immunoassay of claim 11, wherein the detection agent is a tagged anti-human antibody or fragment thereof that binds to human IgG, IgM or IgA antibody or fragment thereof.

14. The immunoassay of claim 11, wherein the capture agent comprises two or more peptides.

15. The immunoassay of claim 11, wherein the capture agent comprises at least one membrane protein peptide selected from Tables 1 and 9 or a peptide having at least 90% sequence similarity to the membrane protein peptide in Tables 1 and 9.

16. (canceled)

17. The immunoassay of claim 16, wherein the secondary antibody comprises a detectable tag, wherein the detectable tag is selected from the group consisting of an enzymatic tag, a biotin tag, a chemiluminescent tag and a fluorescent tag.

18-20. (canceled)

21. The immunoassay of claim 11, wherein the immunoassay is a high throughput assay, preferably a chemiluminescent microparticle immune assay (CMIA) or a chemiluminescent immunoassay (CLIA).

22. The immunoassay of claim 11, wherein the capture agent comprises at least one peptide of the membrane protein from Table 2 and at least one peptide of the spike protein from Table 2.

23-24. (canceled)

25. A method of detecting and distinguishing a subject infected with SARS-CoV-2, subject vaccinated and an uninfected individual comprising
obtaining a sample from a subject;
contacting the sample with the immunoassay of claim 22,
wherein detection of the peptide to the membrane protein can distinguish between a subject having had SARS-CoV-2 and a subject vaccinated.

26. A vaccine composition comprising an adjuvant and one or more peptides selected from Tables 1 and 2 or a peptide having at least 90% sequence similarity to one or more peptides in Tables 1, 2 and 9-12.

27-50. (canceled)

51. The method of claim 1, the method comprising:
a) contacting the sample with an antibody that binds to one or more peptides or epitopes selected from SEQ ID NO:1-16 and Tables 1, 2, 6, and 9-12, and
b) detecting the presence of a sample peptide bound to the antibody, wherein the presence of a sample peptide detects SARS-CoV-2 virus in the sample.
